(19) United States
(12) Patent Application Publication
(10) Pub. No.: US 2024/0382463 A1
(43) Pub. Date: Nov. 21, 2024

(54) Composition Comprising HDAC Inhibitor and Immune Checkpoin Inhibitor and Methods For Using the Same

(71) Applicant: Avstera Therapeutics Corp., Malvern, PA (US)

(72) Inventors: Karthik Musunuri, Chester Springs, PA (US); Damian Kovalovsky, Potomac, MD (US); Dileep Kumar, Spring City, PA (US)

(73) Assignee: Avstera Therapeutics Corp., Malvern, PA (US)

(21) Appl. No.: 18/666,657

(22) Filed: May 16, 2024

Related U.S. Application Data
(60) Provisional application No. 63/502,973, filed on May 18, 2023.

(51) Int. Cl. A61K 31/42; A61K 39/00; A61P 35/00; C07K 16/28

(52) U.S. Cl. CPC A61K 31/42; A61P 35/00; C07K 16/2818; C07K 16/2827; A61K 2039/505

(57) ABSTRACT

The present disclosure provides a composition comprising a histone deacetylase (HDAC) inhibitor and an immune checkpoint inhibitor and methods for using the same in treating cancer.

### CROSS-REFERENCE TO RELATED APPLICATIONS

This application claims the priority benefit of U.S. Provisional Application No. 63/502,973, filed May 18, 2023, which is incorporated herein by reference in its entirety.

### REFERENCE TO AN ELECTRONIC SEQUENCE LISTING

The contents of the following electronic sequence listing are incorporated herein by reference in its entirety:
Name of the XML File: AVS-000300US.xml;
File Size: 25,119 bytes;
Date of Creation: 16 May 2024.

### FIELD OF THE DISCLOSURE

The present disclosure relates to a composition comprising a histone deacetylase (HDAC) inhibitor and an immune checkpoint inhibitor and methods for using the same. In particular, the composition is useful in treating cancer.

### BACKGROUND OF THE DISCLOSURE

Checkpoint inhibitors or immune checkpoint inhibitors (“ICIs”) enhance the action of the immune system against tumor cells and have been used in treating cancer. ICIs work by inter alia blocking certain proteins on immune cells called checkpoint proteins (e.g., blocking negative regulators of T-cells). Exemplary proteins on immune cells that are modulated by ICIs include, but are not limited to, programmed cell death-1 protein (PD-1, also known as cluster of differentiation 279 (CD279)), programmed death ligand-1 protein (PDL-1, also known as cluster of differentiation 274 (CD274)), and cytotoxic T-lymphocyte-associated protein 4 (CTLA-4). These proteins normally help keep the immune system in check, preventing it from attacking healthy cells in the body. However, cancer cells can sometimes take advantage of these checkpoint proteins to evade the immune system and grow unchecked.

By inhibiting checkpoint proteins, checkpoint inhibitors can allow the immune system to recognize and attack cancer cells more effectively. This can lead to improved outcomes for some types of cancer, particularly those that are difficult to treat with traditional chemotherapy or radiation therapy. Exemplary checkpoint inhibitors that have been approved in cancer treatment include ipilimumab, nivolumab, pembrolizumab, atezolizumab, avelumab, durvalumab, cemiplimab, etc. These ICIs target CTLA-4 and/or PD-1/PDL1 receptors.

Unfortunately, however, efficacy of current ICIs in treating cancer is relatively low. Moreover, use of ICIs in treating cancer has shown to lead to adverse events due to the disruption of immunologic homeostasis and the augmentation of immune system response. Some of the common side-effects of checkpoint inhibitors include, but are not limited to, diarrhea, fatigue, cough, nausea, skin rash, poor appetite, constipation, and muscle and joint pain. Checkpoint inhibitors can also disrupt the normal working of various organs, such as the liver, kidneys and hormone making glands (e.g., the thyroid). Disruption of these organs can have serious consequences. Furthermore, response rates for ICIs are only between 20-40% among cancer types in which checkpoint blockade is an approved treatment. Without being bound by any theory, it is believed that this relatively low response rate is most likely due to immunosuppressive mechanisms in the tumor microenvironment that cannot be reversed by the checkpoint inhibitor therapy.

Therefore, there is a need to improve efficacy of checkpoint inhibitors in treating cancer. There is also a need to reduce the occurrence of adverse events (e.g., side-effects) of using checkpoint inhibitors in cancer treatment.

### SUMMARY OF THE DISCLOSURE

Some aspects of the present disclosure are based on the discovery by the present inventors of surprisingly and unexpectedly results that combining an immune checkpoint inhibitor with an HDAC inhibitor increases effectiveness of cancer or solid tumor treatment. Thus, one particular aspect of the disclosure provides a composition comprising a histone deacetylase (HDAC) inhibitor and a checkpoint inhibitor.

In some embodiments, the HDAC inhibitor is an HDAC6 inhibitor or HDAC6 selective inhibitor. Still in other embodiments, the HDAC inhibitor is AMES-negative. In one particular embodiment, the HDAC inhibitor is HDAC6 selective inhibitor that is AMES-negative. In further embodiments, the HDAC inhibitor of the disclosure comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof. In one particular embodiment, the HDAC inhibitor comprises 5-(2-(3,4-dichlorobenzamido)ethyl)-N-hydroxyisoxazole-3-carboxamide. It should be appreciated that the scope of the disclosure is not limited to these particular HDAC inhibitors. In fact, the scope of the disclosure includes any and all HDAC inhibitors that are or can be used in treating a subject suffering from cancer.

Yet in other embodiments, the checkpoint inhibitor comprises a programmed death-1 (PD1) inhibitor, a programmed death ligand-1 (PDL1) inhibitor, a cytotoxic T-lymphocyte-associated protein 4 (CTLA4) inhibitor, a lymphocyte activation gene-3 (LAG-3, or CD223) inhibitor, a T cell immunoreceptor with Ig and ITIM domains (TIGIT) inhibitor, a CD40 inhibitor, an indoleamine 2,3-dioxygenase (IDO) inhibitor, a CCR4 inhibitor, a stimulator of interferon genes (STING) inhibitor, a CD137 inhibitor, a B7-1 inhibitor, a B7-2 inhibitor, or a combination thereof. In one particular embodiment, the checkpoint inhibitor comprises ipilimumab, nivolumab, pembrolizumab, atezolizumab, avelumab, durvalumab, cemiplimab, or a combination thereof. It should be appreciated that the scope of the disclosure is not limited to these particular checkpoint inhibitors. In fact, the scope of the disclosure includes any and all checkpoint inhibitors that are or can be used in treating a subject suffering from cancer.

In further embodiments, the mole ratio of said HDAC inhibitor to said checkpoint inhibitor ranges from 100:1 to about 0.01:1.

Another aspect of the disclosure provides a method for treating a subject suffering from cancer, said method comprising administering a therapeutically effective amount of a histone deacetylase (HDAC) inhibitor and a checkpoint inhibitor. Suitable HDAC inhibitors and the checkpoint inhibitors are those disclosed herein. In some embodiments, said histone deacetylase (HDAC) inhibitor and said checkpoint inhibitor are administered as a single composition. Still in other embodiments, the histone deacetylase (HDAC) inhibitor and said checkpoint inhibitor are administered separately. When administered separately, said histone deacetylase (HDAC) inhibitor and said checkpoint inhibitor are administered within an hour, typically within 3 h, often within 6 h, more often within 12 h, still more often within 24 h, yet more often within 2 days, and most often within 5 days from one another. Alternatively, HDAC inhibitor and the checkpoint inhibitor can be administered in a staggered manner, e.g., a first treatment may be with an HDAC inhibitor or a checkpoint inhibitor and the next treatment can be with the other. In this manner, one can alternate administration of an HDAC inhibitor and a checkpoint inhibitor. However, it should be appreciated that the scope of the disclosure is not limited to these treatment protocols. In fact, the scope of the disclosure includes all possible combinations and permutations of treatment protocol as long as both HDAC inhibitor and checkpoint inhibitor are used in treating the subject suffering from cancer.

In some embodiments, the HDAC inhibitor is an HDAC6 inhibitor. Yet in other embodiments, the HDAC inhibitor is AMES-negative. In one particular embodiment, the HDAC inhibitor is HDAC6 selective inhibitor that is AMES-negative.

Still in other embodiments, said HDAC inhibitor comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof. In one particular embodiment, said HDAC inhibitor comprises 5-(2-(3,4-dichlorobenzamido)ethyl)-N-hydroxyisoxazole-3-carboxamide.

Yet in other embodiments, said checkpoint inhibitor comprises a programmed death-1 (PD1) inhibitor, a programmed death ligand-1 (PDL1) inhibitor, a cytotoxic T-lymphocyte-associated protein 4 (CTLA4) inhibitor, or a combination thereof. In one particular embodiment, said checkpoint inhibitor comprises ipilimumab, nivolumab, pembrolizumab, atezolizumab, avelumab, durvalumab, cemiplimab, or a combination thereof.

In further embodiments, a mole ratio of said HDAC inhibitor to said checkpoint inhibitor administered ranges from 100:1 to about 0.01:1.

In other embodiments, said cancer is a solid tumor cancer. Still in other embodiments, said cancer comprises melanoma, non-Hodgkin's lymphoma, Hodgkin's disease, Ewing's sarcoma, testicular cancer, prostate cancer, ovarian cancer, bladder cancer, larynx cancer, cervical cancer, nasopharynx cancer, breast cancer, colon cancer, pancreatic cancer, head and neck cancer, esophageal cancer, rectal cancer, small-cell lung cancer, non-small cell lung cancer, brain tumor, merkel-cell carcinoma, mesothelioma, and other CNS neoplasm.

Other aspects of the disclosure provide use of a composition comprising a histone deacetylase (HDAC) inhibitor and a checkpoint inhibitor in preparation for a medicament for treating solid tumors.

In some embodiments, said HDAC inhibitor is AMES-negative. Yet in other embodiments, said HDAC inhibitor is a HDAC6 inhibitor. In one particular embodiment, the HDAC inhibitor is HDAC6 selective inhibitor that is AMES-negative. In further embodiments, HDAC inhibitor comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof. In one particular embodiment, said HDAC inhibitor comprises 5-(2-(3,4-dichlorobenzamido)ethyl)-N-hydroxyisoxazole-3-carboxamide.

Still in other embodiments, said checkpoint inhibitor comprises a programmed death-1 (PD1) inhibitor, a programmed death ligand-1 (PDL1) inhibitor, a cytotoxic T-lymphocyte-associated protein 4 (CTLA4) inhibitor, a lymphocyte activation gene-3 (LAG-3, or CD223) inhibitor, a T cell immunoreceptor with Ig and ITIM domains (TIGIT) inhibitor, a CD40 inhibitor, an indoleamine 2,3-dioxygenase (IDO) inhibitor, a CCR4 inhibitor, a stimulator of interferon genes (STING) inhibitor, a CD137 inhibitor, a B7-1 inhibitor, a B7-2 inhibitor, or a combination thereof. Still in other embodiments, said checkpoint inhibitor comprises a programmed death-1 (PD1) inhibitor. In one particular embodiment, said checkpoint inhibitor comprises ipilimumab, nivolumab, pembrolizumab, atezolizumab, avelumab, durvalumab, cemiplimab, or a combination thereof.

Yet in other embodiments, a mole ratio of said HDAC inhibitor to said checkpoint inhibitor ranges from 100:1 to about 0.01:1.

Still another aspect of the disclosure provides a composition comprising a histone deacetylase-6 (HDAC6) inhibitor and a programmed death-1 (PD1) inhibitor. In some embodiments, said HDAC6 inhibitor is AMES-negative. Yet in other embodiments, said HDAC6 inhibitor comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof. In one particular embodiment, said HDAC6 inhibitor comprises 5-(2-(3,4-dichlorobenzamido)ethyl)-N-hydroxyisoxazole-3-carboxamide. Still in other embodiments, said PD1 inhibitor comprises ipilimumab, nivolumab, pembrolizumab, atezolizumab, avelumab, durvalumab, cemiplimab, or a combination thereof. In one particular embodiment, said PD1 inhibitor comprises pembrolizumab. In further embodiments, a mole ratio of said HDAC inhibitor to said checkpoint inhibitor ranges from 100:1 to about 0.01:1.

In further aspect of the disclosure, use of a composition comprising a histone deacetylase-6 (HDAC6) inhibitor and a programmed death-1 (PD1) inhibitor for treating a solid tumor in a subject is provided. In some embodiments, said solid tumor is a solid tumor cancer. Still in other embodiments, said solid tumor comprises melanoma, non-Hodgkin's lymphoma, Hodgkin's disease, Ewing's sarcoma, testicular cancer, prostate cancer, ovarian cancer, bladder cancer, larynx cancer, cervical cancer, nasopharynx cancer, breast cancer, colon cancer, pancreatic cancer, head and neck cancer, esophageal cancer, rectal cancer, small-cell lung cancer, non-small cell lung cancer, brain tumors, other CNS neoplasms. In further embodiments, said solid tumor comprises breast cancer, colon cancer, or melanoma.

### BRIEF DESCRIPTION OF THE DRAWINGS

FIG. 1A is Western blot analysis of Raw264.7 murine macrophages unpolarized (M0) or after polarization by stimulation with IFN-γ plus LPS (M1) in the absence or presence of AVS100.

FIG. 1B is Western blot analysis of BMA31A7 murine macrophages unpolarized (M0) or after polarization by stimulation with IL-4 plus IL-13 (M2) in the absence or presence of AVS100.

FIG. 1C is Western blot analysis of BMDM polarized toward M1 and M2 conditions in the absence or presence of AVS100.

FIG. 1D shows qPCR analysis for the indicated genes in BMDM polarized towards M1 and M2 phenotypes. n=3, ****p<0.0001 unpaired T-test.

FIG. 1E shows FACs analysis of F4/80 gated macrophages after polarization of BMDM towards M1 and M2 in the absence or presence of AVS100. Data is representative of 2 to 3 independent experiments.

FIG. 2A is qPCR analysis of primary human macrophages after stimulation with IFN-γ plus LPS (M1) or IL-4 (M2) in the absence or presence of AVS100 to identify expression of M1 and M2 markers. Data is representative of two experiments. n=3, * p<0.05, **p<0.01 unpaired T-test.

FIG. 2B is Principal Component Analysis of human macrophages polarized towards M1 and M2 phenotype in the absence or presence of AVS100.

FIG. 2C shows average gene fold-change by AVS100 treatment within M0, M1 and M2 treatment conditions.

FIG. 2D is heatmaps of all genes showing at least two-fold upregulation (M1 vs M0) or (M2 vs M0).

FIG. 2E shows Trust pathway analysis of genes that were upregulated in M1 or M2 conditions and downregulated by AVS100, showing transcription factor networks that were significantly affected by AVS100 in M1 and M2 polarizing conditions. Significance of the 2-3 transcription factors affected is shown.

FIG. 2F is heatmaps representing gene regulation identified in FIG. 2E by AVS100 treatment.

FIG. 2G is Western blot analysis after AVS100 treatment of myeloid and lymphoid human cell lines.

FIG. 2H is quantification of western blot shown in FIG. 2G.

FIG. 2I is Western blot analysis of THP1 cells differentiated to macrophages, unpolarized or polarized to M2 in the absence or presence of AVS100.

FIG. 2J is quantification of western blot shown in FIG. 2I. Western blots are representative of 2-3 independent experiments.

FIG. 3 is Western-blot analysis of THP1 macrophages stimulated with vehicle (M0) or IL4 (M2) for the indicated times in the presence or absence of a HDAC6 selective inhibitor of the disclosure.

FIG. 4 is a table showing comparison of plasma pharmacokinetic parameters of a HDAC6 selective inhibitor of the disclosure in sodium CMC suspension and PEG6000 formulation following a single oral administration in fed and fasted male C57BL/6 mice.

FIG. 5A is analysis of tumor volume in the SM1 melanoma model by daily oral gavage of AVS100 at the indicated doses.

FIG. 5B is analysis of tumor volume in the SM1 melanoma model by anti-PD1 injection i.p. three times a week and daily oral gavage of AVS100. Each treatment group corresponds to 10-13 mice. **** p<0.0001, ** p<0.01 Two-way anova with Turkey post-test.

FIG. 5C shows images of dissected tumors at endpoint.

FIG. 5D is tumor volume of individual mice per treatment group. In brackets is represented the percentage of responder mice (R) having the specified tumor volume at endpoint.

FIG. 5E shows percentage of intratumoral immune cells as obtained by FACs analysis. Data corresponds to 6-7 mice per group. Only the significance of AVS100 vs Control and anti-PD1 plus AVS100 vs anti-PD1 treatments are represented. * p<0.05 unpaired T-test.

FIG. 5F is FACs analysis of the TAM phenotype. Numbers represent the percentage of events within each gate.

FIG. 5G is quantitation of the percentage of M1, M2 and M1/M2 ratio as analyzed in FIG. 5F. Data corresponds to 6-7 mice per group. Only the significance of AVS100 vs Control and anti-PD1 plus AVS100 vs anti-PD1 treatments are represented. * p<0.05, **p<0.01 unpaired T-test.

FIG. 6A shows analysis of tumor volume in the CT26 colon cancer model by anti-PD1 injection i.p. three times a week and daily oral gavage of AVS100. Each treatment group corresponds to 15 mice. *** p<0.001 Two-way anova with Turkey post-test.

FIG. 6B shows tumor volume of individual mice per treatment group. In brackets is represented the percentage of responder mice having the specified tumor volume at endpoint.

FIG. 6C is quantitation of the percentage of M1, M2 and M1/M2 ratio. Data corresponds to 6-7 mice per group. Only the significance Only the significance of AVS100 vs Control and anti-PD1 plus AVS100 vs anti-PD1 treatments are represented. * p<0.05, **p<0.01 unpaired T-test.

FIG. 7A is cluster analysis of tumor infiltrating immune cells (CD45⁺) identifying different immune cell types. Lymphoid corresponds to B, T and NKT cells. Myeloid corresponds to basophils, monocytes, macrophages, neutrophils, and dendritic cells.

FIG. 7B is cluster analysis of TAM subclusters present in all treatment groups.

FIG. 7C shows expression of M1 (top half) and M2 (bottom half) genes in each of the TAM sub-clusters.

FIG. 7D is a table showing functional classification of TAM sub-clusters based on specific gene expression.

FIG. 7E shows cluster analysis representing TAM sub-clusters in treatment groups.

FIG. 7F shows proportional changes in TAM sub-clusters in treatment groups.

FIG. 7G is a heatmap representing single-cell levels of selected genes representing T-cell signal and effector function in T-cells.

FIG. 7H is a heatmap representing single-cell levels of selected genes representing the NFKB pathway in T-cells.

FIG. 7I is histograms comparing mRNA levels of selected T-cell signaling/effector genes in the indicated conditions.

FIG. 8A shows proportion of infiltrating immune cell types in SM1 melanoma tumors as identified by scRNAseq analysis at the endpoint of the indicated treatments.

FIG. 8B shows Elsevier pathway analysis of genes that were upregulated by AVS100 treatment in tumor infiltrating T-cells in the presence or absence of anti-PD1 treatment of mice.

FIG. 8C shows pairwise comparison of T-cell effector genes in T-cells, showing upregulation by an HDAC6 selective inhibitor of the disclosure.

FIG. 9A shows tumor growth on mice that have achieved complete remission and were re-implanted one month after termination of treatment. Control (CTR) corresponds to naïve mice that have not received tumor before the time of re-engraftment. Empty arrows indicate dissection of tissues for TCRseq analysis.

FIG. 9B shows phenotype of CD8 T-cells from tumors and spleens to identify CM (CD62L⁺CD44⁺) and EM (CD62L⁻CD44⁺) phenotypes.

FIG. 9C is analysis of the clonality index (1-Pielou) based on TCRβ sequencing of CD8 CM+EM subsets. Each dot represents an individual mouse from the indicated groups.

FIG. 9D is a pie chart representing the proportion of the most immunodominant T-cell clones in each treatment group.

FIG. 9E shows absolute counts of the 10 immunodominant T-cell clones in each treatment group.

FIG. 10 is a table showing summary of evaluating safety and toxicology of an HDAC6 selective inhibitor of the disclosure.

### DETAILED DESCRIPTION OF THE DISCLOSURE

The terms “comprise(s),” “include(s),” “having,” “has,” “can,” “contain(s),” and variants thereof, as used herein, are intended to be open-ended transitional phrases, terms, or words that do not preclude the possibility of additional steps or components or ingredients. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. Accordingly, the transitional phrases “consisting of” and “consisting essentially of” may be interpreted to be subsets of the open-ended transitional phrases, such as “comprising” and “including,” such that any use of an open-ended phrase to introduce a recitation of a series of elements, limitations, components, ingredients, materials, or steps should be interpreted to also disclose recitation of the series of elements, limitations, components, ingredients, materials, or steps using the closed terms “consisting of” and “consisting essentially of.” For example, the recitation of a composition “comprising” components A, B and C should be interpreted as also disclosing a composition “consisting of” components A, B, and C as well as a composition “consisting essentially of” components A, B, and C.

Tumor cells can evade destruction by the immune system by triggering immune checkpoint receptors, such as cytotoxic T-lymphocyte-associated protein 4 (CTLA-4), programmed cell death protein 1 (PD-1), or programmed death-ligand 1 (PD-L1), that are expressed on T-cells and whose engagement inhibit T-lymphocyte function. Immune checkpoint inhibitors (i.e., checkpoint inhibitors or ICIs) are molecules that prevent this immunosuppression by blocking the engagement of these checkpoint molecules, thereby reinvigorating the antitumor immune response. Currently, checkpoint inhibitors have been approved for use in several types of cancer, including lung cancer, melanoma, bladder cancer, and kidney cancer, among others.

Unfortunately, enhancing the immune response using checkpoint inhibitors can result in immune-related adverse events that can affect nearly every organ system. Moreover, toxicities are common and some studies have shown they occur in as many as 72% of patients who are treated with checkpoint inhibitors. See, cancercareontario.ca/en/guidelines-advice/types-of-cancer/52976. Toxicities can range from mild to severe. Some of the side-effects of using ICIs in cancer treatment include, but are not limited to, dermatological toxicities; diarrhea and colitis; endocrinopathies; hepatic toxicities; neurotoxicities; pneumonitis; renal toxicities; ocular toxicities; hematological toxicities; inflammatory arthritis; oral toxicities; and cardiotoxicity. Of these, common side effects of checkpoint inhibitors include, but are not limited to, fatigue, skin rash, diarrhea, and joint pain. Less common but more serious side effects can include inflammation of the lungs, liver, or other organs, which can be life-threatening in some cases.

Checkpoint inhibitors that are currently approved for cancer treatment include anti-PD1 antibodies that block the PD-1 receptor on T cells. PD-1 is an immune checkpoint protein that is often overexpressed on cancer cells, and when it binds to its ligand, PD-L1, it can inhibit the activity of T cells and prevent them from attacking cancer cells. By blocking the interaction between PD-1 and PD-L1, anti-PD-1 molecules can activate the immune system to recognize and attack cancer cells. Other checkpoint inhibitors that are useful in treating cancer include, but are not limited to, programmed death-1 (PD1) inhibitors, programmed death ligand-1 (PDL1) inhibitors, cytotoxic T-lymphocyte-associated protein 4 (CTLA4) inhibitors, as well as other checkpoint inhibitors that are known to one of skill in the art. Exemplary checkpoint inhibitors that are useful in the present disclosure include, but are not limited to, nivolumab, pembrolizumab, toripalimab (humanized IgG4 monoclonal antibody), pidilizumab, BMS-936559 (Bristol Myers Squibb), atezolizumab, avelumab, ipilimumab, atezolizumab, avelumab, durvalumab, cemiplimab, vopratelimab, spartalizumab, camrelizumab, sintilimab, tislelizumab, INCMGA00012 (MGA012), AMP-224, AMP-514, acrixolimab, KN035, cosibelimab, AUNP12, CA-170, BMS-986189, retifanlimab, dostarlimab, and other checkpoint inhibitors that are being or will be developed. In general, any checkpoint inhibitors can be used in the compositions and methods of the present disclosure.

In general, however, any immune checkpoint inhibitors can be used in the present disclosure including, but not limited to, PD-1 inhibitors, PD-L1 inhibitors, CTLA-4 inhibitors, LAG3 inhibitors, TIM3 inhibitors, cd47 inhibitors, and B7-H1 inhibitors. Thus, in one aspect, the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In some embodiments, the immune checkpoint inhibitor is a programmed cell death protein (PD-1) inhibitor. PD-1 is a T-cell coinhibitory receptor that plays one of the key roles in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Exemplary antibodies that bind to PD-1 (i.e., anti-PD-1 antibodies) include, but are not limited to, nivolumab, pembrolizumab, STI-A1014, and pidilzumab. For a general discussion of the availability, methods of production, mechanism of action, and clinical studies of anti-PD-1 antibodies (i.e., antibodies that bind to PD-1), see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer, 2014, 111, pp. 2214-19.

Still in other embodiments, the immune checkpoint inhibitor is a PD-L1 (also known as B7-H1 or CD274) inhibitor. Examples of PD-L1 inhibitors include antibodies that specifically bind to PD-L1 (i.e., anti-PD-L1 antibodies). Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. Patent Application Publication Nos. 2014/0341917, and 2013/0071403, PCT Patent Application Publication No. WO 2015036499, and Naido et al., British Journal of Cancer, 2014, 111, pp. 2214-19.

Yet in other embodiments, the immune checkpoint inhibitor is a CTLA-4 inhibitor. CTLA-4, also known as cytotoxic T-lymphocyte antigen 4, is a protein receptor that downregulates the immune system. CTLA-4 binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4 (i.e., anti-CTLA-4 antibodies). Particular anti-CTLA-4 antibodies include, but are not limited to, ipilimumab and tremelimumab. Anti-CTLA-4 antibodies are well known to one of skill in the art. See, for example, U.S. Pat. Nos. 6,984,720 and 6,207,156, and Naido et al., British Journal of Cancer, 2014, 111, pp. 2214-19.

In further embodiments, the immune checkpoint inhibitor is a LAG3 inhibitor. LAG3, Lymphocyte Activation Gene 3, is a negative co-stimulatory receptor that modulates T cell homeostasis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. See, for example, U.S. Patent Application Publication No. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. Patent Application Publication Nos. 2011/0150892, U.S. 2014/0093511, U.S. 20150259420, and Huang et al., Immunity, 2004, 21, pp. 503-13.

Still yet in other embodiments, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Examples of TIM3 inhibitors include antibodies that specifically bind to TIM3. See, for example, U.S. Patent Application Publication Nos. 2015/0225457 and 2013/0022623, U.S. Pat. No. 8,522,156, Ngiow et al., Cancer Res. 2011, 71, pp. 6567-71, Ngiow, et al., Cancer Res, 2011, 71, pp. 3540-51, and Anderson, Cancer Immunology Res, 2014, 2, pp. 393-98.

In another aspect, the immune checkpoint inhibitor is a CD47 inhibitor. See Unanue, E. R., PNAS, 2013, 110, pp. 10886-87.

The term “antibody” includes intact monoclonal antibodies, polyclonal antibodies, and multispecific antibodies formed from at least two intact antibodies, so long as they exhibit the desired biological activity. In one aspect, the antibodies are humanized monoclonal antibodies made by means of recombinant genetic engineering.

Other suitable immune checkpoint inhibitors of the disclosure include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845. Another examples of suitable immune checkpoint inhibitors of the disclosure include compounds with peptide moieties that inhibit PD-1 signaling. Examples of such compounds are disclosed in U.S. Pat. No. 8,907,053. Still other suitable class of immune checkpoint inhibitors of the disclosure include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer, 2012, 12, pp. 252-64; Löb, Cancer Immunol Immunother, 2009, 58, pp. 153-57. Particular IDO blocking agents include, but are not limited to levo-1-methyl typtophan (L-1MT) and 1-methyl-tryptophan (1MT). Qian et al., Cancer Res, 2009, 69, pp. 5498-504; and Löb et al., Cancer Immunollmmunother, 2009, 58, pp. 153-57.

Histone deacetylases (HDACs) are a family of proteins involved in the epigenetic regulation of target genes through deacetylating lysine F-amino groups on histone tails to promote a status of DNA condensation and transcriptional silencing. HDACs have been found to modify other nonhistone proteins, and these modifications can influence a variety of cellular functions without modifying the acetylation of the chromatin. It is widely established that epigenetic modifications are important mechanisms triggering cancer development and progression, and histone modifications represent a versatile set of epigenetic signs that are involved in dynamic cellular processes, such as transcription and DNA repair, as well as in the stable maintenance of chromatin structure. Changes in modifications of histones have been linked to unusual expression of many genes which leads to cancer development and progression.

A total of eighteen known mammalian HDACs have been identified. These HDACs can be classified into four subgroups according to their homology to yeast enzymes: class I (HDAC1, 2, 3, and 8), class IIa (HDAC4, 5, 7, and 9), class IIb (HDAC6 and HDAC10), class III or sirtuins (SIRT 1-7), and class IV (HDAC11). In comparison with other HDAC family members, HDAC6 is relatively unique. It contains two tandem deacetylase catalytic domains (CD1 and CD2) and is primarily localized to the cytosol, and its preferred substrates include a variety of nonhistone proteins, such as α-tubulin, cortactin, HSP-90, and HSF-1. HDAC6 promotes the proliferation and metastasis of melanoma cells, and knockdown of HDAC6 decreases proliferation and induces the G1-cell cycle arrest of melanoma cells. Moreover, HDAC6 increases the protein level of tyrosine-protein phosphatase nonreceptor type 1 (PTPN1), which is responsible for promoting proliferation, colony formation, and migration while decreasing the apoptosis of melanoma cells through activating extracellular signal-regulated kinase 1/2 (ERK1/2). HDAC6 is also a modulator of the expression of specific tumor-associated antigens, MHC class I, and costimulatory molecules in melanoma. Selective pharmacological inhibition or genetic abrogation of HDAC6 has been shown to play a critical role in the immune checkpoint blockade by downregulating the expression of PD-L1, which exhibits an opposite effect relative to class I and pan-HDAC inhibitors.

HDAC inhibitors have been extensively studied in cancer treatment. HDAC inhibitors work by inhibiting the activity of histone deacetylases, which are enzymes that remove acetyl groups from histones. This modification can result in tighter packaging of DNA around histones, making genes less accessible for transcription. By inhibiting HDACs, the expression of genes that promote cell death and differentiation can be restored, leading to a reduction in cancer cell growth and proliferation.

Both HDAC inhibitors and checkpoint inhibitors (such as anti-PD-1 antibodies) are used in cancer treatment, but they work through different mechanisms and can be used to treat different types of cancer. For example, HDAC inhibitors are commonly used to treat hematological malignancies, such as lymphoma and leukemia, while anti-PD-1 antibodies are used to treat solid tumors, such as melanoma and lung cancer. In some cases, these drugs may be used in combination with other therapies, such as chemotherapy or radiation, to enhance their effectiveness. However, to date no one has recognized benefits of combining an HDAC inhibitor and a checkpoint inhibitor in treating cancer.

Surprisingly and unexpectedly, the present inventors have discovered that combining an ICI with an HDAC inhibitor significantly increases effectiveness of cancer or solid tumor treatment. In some embodiments, HDAC inhibitors of the disclosure are HDAC6 selective inhibitors. The terms “HDAC6 inhibitor,” “HDAC6 selective inhibitor,” and “HDAC6 specific inhibitor” are used interchangeably herein and refers to HDAC inhibitors that selectively inhibit HDAC6 compared to other isoforms of HDAC. In some embodiments, HDAC6 inhibitors have inhibition activity for HDAC6 of at least about fifty times (50×), generally at least about one-hundred times (100×), typically at least about 250×, often at least about 500×, more often at least about 1,000×, and most often at least about 2,000× compared to inhibitory activity over other HDAC isoforms. In one particular embodiment, HDAC inhibitors of the disclosure have HDAC6 selectivity of at least about 100×, typically at least about 500×, often at least about 1,000×, more often at least about 2,000×, and most often at least about 3,000× compared to its inhibitory activity to HDAC9 isoform.

Accordingly, some aspects of the disclosure provide compositions and methods to overcome the possibility of poor outcomes and/or reduce or eliminate the severity or the incidences of side-effects from using a checkpoint inhibitor in cancer treatment by including a histone deacetylase (HDAC) inhibitor. Thus, one particular aspect of the disclosure provides a composition comprising a HDAC inhibitor and a checkpoint inhibitor as well as a method for using the same in treating cancer. Exemplary HDAC inhibitors that can be used in the present disclosure include, but are not limited to, any HDAC inhibitors known to one skilled in the art, such as HDAC inhibitors disclosed in PCT Patent Application Publication No. WO 2018/183701, published Oct. 4, 2018, and PCT Patent Application Publication No. WO 2017/040564, published Mar. 9, 2017, all of which are incorporated herein by reference in their entirety. In one particular embodiment, HDAC inhibitor of the disclosure is of the formula:

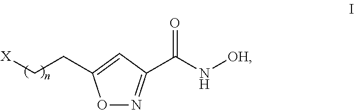

or a pharmaceutically acceptable salt, or solvate thereof, wherein:
X is selected from the group consisting of:

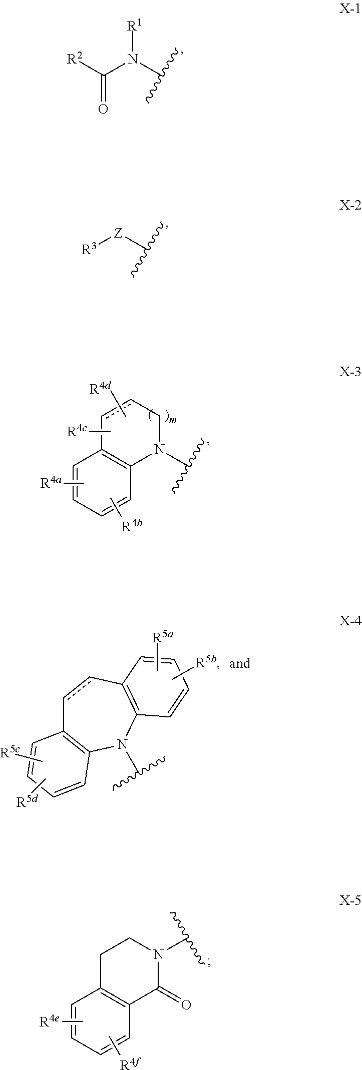

R¹ is selected from the group consisting of hydrogen and C₁₋₄ alkyl;
R² is selected from the group consisting of optionally substituted C₆-C₁₄ aryl and aralkyl;
R³ is selected from the group consisting of optionally substituted C₆-C₁₄ aryl, and optionally substituted 5- to 14-membered heteroaryl;
R^{4a}a, R^{4b}, R^{4e}, and R^{4f} are independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, and haloalkoxy;
R^{4c} and R^{4d} are independently selected from the group consisting of hydrogen and C₁₋₄ alkyl; or
R^{4c} and R^{4d} taken together form a —C(═O)— with the carbon atom to which they are attached;
R^{5a}, R^{5b}, R^{5c}, and R^{5d} are independently selected from the group consisting of hydrogen, halogen, hydroxy, nitro, cyano, —NR^{a}R^{b}, —C(═O)NR^{a}R^{b}, —C(═O)R^{c}, C₁₋₆ alkyl, C₂₋₆ alkenyl, C₂₋₆ alkynyl, C₁₋₆ alkoxy, C₁₋₆ haloalkyl, and haloalkoxy;
Z is selected from the group consisting of —O—, and —N(R⁸)—; or
Z is absent;
R⁸ is selected from the group consisting of hydrogen, C₁₋₄ alkyl, optionally substituted C₃₋₆ cycloalkyl, optionally substituted C₆-C₁₄ aryl, aralkyl, optionally substituted 5- to 14-membered heteroaryl, and heteroaralkyl;
m is 0, 1, or 2;
n is 1, 2, 3, 4, 5, or 6;
[symbol] represents a single or double bond;
R^{a} and R^{b} are independently selected from the group consisting of hydrogen, C₁₋₆ alkyl, optionally substituted C₃₋₆ cycloalkyl, optionally substituted C₆-C₁₄ aryl, optionally substituted 5- to 14-membered heteroaryl; or
R^{a} and R^{b} taken together with the nitrogen atom to which they are attached form an optionally substituted 3- to 12-membered heterocyclo; and
R^{c} is C₁₋₄ alkyl,
with the proviso that when Z is absent, R³ is a bicyclic or tricyclic C₁₀₋₁₄ aryl, a bicyclic or tricyclic 9- to 14-membered heteroaryl.

Other suitable HDAC inhibitors of the disclosure include HDAC6 inhibitors disclosed in Shen and Kozikowski, Expert Opinion on Therapeutic Patents 2020, 30, pp. 121-136; Bergman et al., J Med Chem., 2012, 55, pp. 9891-9899; PCT Patent Application Publication Nos. WO 2014/072714; WO 2016/067040; WO 2016/190630; WO 2019/139921; WO 2015/054474; WO 2017/075192; WO 2018/089651; WO 2014/181137; WO 2016/067038; WO 2017/208032; WO 2016/168598; WO 2016/168660; WO 2017/218950; WO 2014/178606; WO 2015/087151; WO 2015/102426; WO 2015/137750; WO 2018/189340; WO 2018/130155; WO 2017/222950; WO 2017/222951; WO 2017/222952; WO 2016/031815; WO 2017/014170; WO 2017/014321; WO 2017/033946; WO 2019/027054; WO 2019/166824; WO 2019/110663; WO 2017/018803; WO 2017/018805; WO 2017/023133; WO 2017/065473; and WO 2018/183701; and U.S. Patent Application Publication Nos. US 2015/0239869; 2016/0221973; 2016/0222022; and 2016/0221997.

The present inventors have discovered that by combining an HDAC inhibitor and a checkpoint inhibitor provides heretofore unrecognized synergistic effect, for example, (i) the severity and/or the incidence of side-effects of a checkpoint inhibitor may be reduced or eliminated; and/or (2) the efficacy of cancer treatment is significantly increased compared to using either the HDAC inhibitor or the checkpoint inhibitor alone. In some embodiments, the efficacy of checkpoint inhibitor may be increased by at least about 5%, typically by at least about 10%, often by at least 25%, and most often by at least 50%. Increase in efficacy can be readily determined by one skilled in the art. For example, one can conduct a clinical trial or in vitro or in vivo study using only an ICI or HDAC inhibitor and compare the efficacy results with a similar study using a combination of an ICI and HDAC inhibitor.

The present inventors have found that checkpoint inhibition with an ICI (e.g., anti-PD1, anti-PDL1, and/or anti-CTLA4 antibodies) reverses the tolerant state of T-cells, triggering an effective antitumoral T-cell response. While several checkpoint inhibitors have been approved for a variety of cancers, including lymphoma, melanoma, non-small cell lung cancer, bladder and kidney cancer, and some head and neck cancers, unfortunately, up to 60% of cancer patients remain resistant to immunotherapy in some indications. Given that the immunoregulatory microenvironment of tumors is highly determined by myeloid cells, including macrophages and myeloid-derived suppressor cells, the present inventors have theorized that the highly specific HDAC6 inhibitor (such as AVS100 or SS-208) is a suitable novel therapy for solid tumors by blocking immunoregulatory functions in tumor-associated macrophages (TAMs).

Experiments by the present inventors showed that AVS100 has a standalone antitumoral effect in cancers (such as melanoma and colon cancer models) and also potentiates the antitumoral response of ICI (e.g., anti-PD1) therapy, resulting in the eradication of melanoma and increased ICI sensitivity in colon cancer. In addition, cured mice did not relapse after termination of the treatment and became resistant to a subsequent tumor challenge. The increased antitumoral effect of combined HDAC inhibitor and ICI was associated with increased amplification of immunodominant T-cell clones within the tumor, supporting the present inventors' theory that an HDAC inhibitor changes the tumor microenvironment to make it more permissible for antitumoral T-cell expansion and effector functions.

The increased proinflammatory tumor microenvironment was evidenced by increased M1/M2 TAM balance. Previous scRNAseq analyses of TAMs from diverse cancer patients and mice identified seven dominant TAMs with unique phenotypes. Based on this characterization, administration of HDAC inhibitors led to an increase in proinflammatory IFN-TAMs and a decrease in lipid-associated and regulatory TAMs. ICI treatment led to a similar phenotype, and the combination of an HDAC inhibitor and an ICI treatment had a synergistic effect, highlighting the simultaneous disruption of immunoregulatory signals in T-cells and macrophages. The increase of IFN-TAM by ICI treatment was expected as this phenotype is generated by T-cell derived IFN-g signals, which are increased during an antitumoral T-cell response.

Gene expression analysis in tumor infiltrating T-cells revealed an increase of proinflammatory and NFKB signals after an HDAC inhibitor treatment independently of an ICI treatment, including Stat4, Il12r, Nfkb1, and Rel. Some T-cell effector genes were induced by an HDAC inhibitor treatment but showed no significant enhancement in combination with an ICI. Contrary to the increase in the proinflammatory phenotype in tumor models, analysis of the direct effects of an HDAC inhibitor on macrophages in vitro showed that the HDAC inhibitor exerts a direct role in macrophages by blocking the acquisition of an anti-inflammatory phenotype under M2 polarizing conditions while mildly affecting the acquisition of a M1 phenotype. This indicates that the HDAC inhibitor-mediated inhibition of M2 polarization in macrophages favors a proinflammatory tumor microenvironment in which competing proinflammatory and anti-inflammatory signals coexist in vivo, while these competing signals do not appear to be present during in vitro models of macrophage polarization.

STAT6 activation in response to IL-4 signals triggers the alternative activation of macrophages and M2 polarization, and c-Myc is essential for M2 polarization in macrophages. The HDAC inhibitor did not inhibit STAT6 phosphorylation in response to IL-4 stimulation. It has previously been reported that HDAC6 inhibition leads to acetylation of a-tubulin, inefficient nuclear translocation of c-Myc, and its degradation in B-cell lymphoma. c-Myc acetylation also occurs in response to pan-HDAC inhibition, leading to Trail activation and apoptosis in leukemia. The present inventors have observed that an HDAC inhibitor treatment of macrophages increased acetyl-c-Myc levels and reduced total c-Myc levels, as a contributing mechanism for the impaired M2 polarization.

Contrary to non-specific pan-HDAC inhibitors that have received FDA approval and exert direct toxicity to tumor cells and are AMES test positive, in some embodiments HDAC inhibitors (e.g., ASV100) of the disclosure are AMES-negative, and thus have minimal toxicity or mutagenic potential. The terms “AMES-negative,” “AMES test negative,” and “AMES negative” are used interchangeably herein and refer to an HDAC inhibitor that is negative in AMES test. One particular HDAC inhibitor, ACY-1215 (Ricolinostat® or Rocilinostat®), is an oral bioavailable class I and HDAC6 inhibitor with a ten-fold specificity over HDAC6 than other HDAC isoforms. ACY-1215 has been tested in clinical trials for various indications such as multiple myeloma and lymphoma, and its anti-tumoral actions have been attributed to direct effects in cancer cells. ACY-1215 increased apoptosis in multiple myeloma by inhibiting aggresomal formation and autophagy. It has been shown that ACY-1215 inhibits cell proliferation in cancer cells by disrupting AKT and ERK signaling pathways and activates p53-mediated apoptosis, among other mechanisms. Contrary to AVS100 effects, ACY-1215 reduces proinflammatory functions, most likely due to its direct toxicity on normal lymphoid and myeloid cells.

Yet in other embodiments, HDAC inhibitors of the disclosure have HDAC6 inhibitory selectivity of at least about one-hundred times (100×), typically at least about 250×, often at least about 500×, and more often at least about 1,000× compared to inhibitory activity over other HDAC isoforms. In one particular embodiment, HDAC inhibitors of the disclosure have HDAC6 inhibitory selectivity of at least about 50×, generally at least about 100×, typically at least about 250×, often at least about 500×, more often at least about 1,000×, more often at least about 2,000×, and most often at least about 3,000× compared to its inhibitory activity to HDAC9 isoform.

Alternatively, the IC₅₀ of HDAC inhibitors of the disclosure for HDAC6 is about 0.5 μM or less, typically about 0.1 μM or less, often about 0.05 μM or less, more often about 0.025 μM or less, and most often about 0.01 μM or less. When referring to a numerical value, the terms “about” and “approximately” are used interchangeably herein and refer to being within an acceptable error range for the particular value as determined by one of ordinary skill in the art. Such a value determination will depend at least in part on how the value is measured or determined, e.g., the limitations of the measurement system, i.e., the degree of precision required for a particular purpose. For example, the term “about” can mean within 1 or more than 1 standard deviation, per the practice in the art. Alternatively, the term “about” when referring to a numerical value can mean ±20%, typically ±10%, often ±5% and more often ±1% of the numerical value. In general, however, where particular values are described in the application and claims, unless otherwise stated, the term “about” means within an acceptable error range for the particular value, typically within one standard deviation.

In some embodiments, HDAC inhibitors of the disclosure has a specificity or selectivity for HDAC6 that is at least about 10× or more, typically at least about 25× or more, often at least 50× or more, and still more often at least about 100× or more than ACY-1215. As used herein, the terms “specificity” and “selectivity” are used interchangeably herein and refers to selectivity of a particular HDAC inhibitor for a particular HDAC isoform (e.g., HDAC6) relative to other HDAC isoform. Alternatively, the terms “specificity” and “selectivity” refers to the relative IC₅₀ concentration compared to another HDAC inhibitor, e.g., ACY-1215. It is believed that antitumoral effect of HDAC inhibitors of the disclosure, e.g., AVS100 and Nexturastat-A®, is mediated by inter alia regulating macrophage function and the intratumoral microenvironment.

The present disclosure includes pharmaceutical compositions comprising an HDAC inhibitor and an ICI. It should be appreciated that the HDAC inhibitor and/or ICI can be in the form of a pharmaceutically acceptable salt or solvate thereof. The pharmaceutical composition can also include at least one pharmaceutically acceptable carrier or excipient, and optionally other therapeutic and/or prophylactic ingredients. In some embodiments, the mole ratio of HDAC to ICI ranges from about 100:1 to about 1:100, typically from about 75:1 to about 1:75, often from about 50:1 to about 1:50, more often from about 25:1 to about 1:25, and most often from about 10:1 to about 1:10.

Another aspect of the disclosure provides pharmaceutical compositions comprising HDAC and/or a checkpoint inhibitor of the disclosure, or a pharmaceutically acceptable salt or solvate thereof (hereinafter collectively or singularly referred to as “active pharmaceutical ingredient” or API), together with at least one pharmaceutically acceptable carrier, and optionally other therapeutic and/or prophylactic ingredients.

In general, the API of the disclosure are administered in a therapeutically effective amount by any of the accepted modes of administration for agents that serve similar utilities. Suitable dosage ranges are typically about 1 mg to about 5 g daily, typically about 1 mg to about 2 g daily, often about 1 mg to about 1 g daily, more often about 1 mg to 500 mg daily, still more often from about 1 mg to about 100 mg daily, and most often about 1 mg to about 30 mg daily, depending on numerous factors such as the severity of the disease to be treated, the age and relative health of the subject, the potency of the API used, the route and form of administration, the indication towards which the administration is directed, and the preferences and experience of the medical practitioner involved. One of ordinary skill in the art of treating such diseases is typically able, without undue experimentation and in reliance upon personal knowledge and the disclosure of this application, to ascertain a therapeutically effective amount of the API of the disclosure.

Typically, API of the disclosure is administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. One particular manner of administration is oral using a convenient daily dosage regimen which can be adjusted according to the degree of affliction.

API of the disclosure, together with one or more conventional adjuvants, carriers, or diluents, can be placed into the form of pharmaceutical compositions and unit dosages. The pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

API of the disclosure can be formulated in a wide variety of oral administration dosage forms. The pharmaceutical compositions and dosage forms can comprise API of the disclosure or pharmaceutically acceptable salts thereof as the active component. The pharmaceutically acceptable carriers can be either solid or liquid. Solid form preparations include powders, tablets, pills, capsules, cachets, suppositories, and dispersible granules. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of API. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of API with encapsulating material as carrier, providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

API of the disclosure can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water.

API of the disclosure can be formulated for topical administration to the epidermis as ointments, creams or lotions, or as a transdermal patch. Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

API of the disclosure can be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

API of the disclosure can also be formulated for vaginal administration. Pessaries, tampons, creams, gels, pastes, foams or sprays containing in addition to the active ingredient such carriers as are known in the art to be appropriate.

API of the disclosure can be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

API of the disclosure can be formulated for aerosol administration, particularly to the respiratory tract and including intranasal administration. API will generally have a small particle size for example of the order of five (5) microns or less. Such a particle size can be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol can conveniently also contain a surfactant such as lecithin. The dose of drug can be controlled by a metered valve. Alternatively, the active ingredients can be provided in a form of a dry powder, for example, a powder mix of API in a suitable powder base such as lactose, starch, starch derivatives such as hydroxypropylmethyl cellulose and polyvinylpyrrolidine (PVP). The powder carrier typically forms a gel in the nasal cavity. The powder composition can be presented in unit dose form, for example, in capsules or cartridges of e.g., gelatine or blister packs from which the powder can be administered by means of an inhaler.

When desired, formulations can be prepared with enteric coatings adapted for sustained or controlled release administration of the active ingredient. For example, API of the disclosure can be formulated in transdermal or subcutaneous drug delivery devices. These delivery systems are advantageous when sustained release of API is necessary or desired and when patient compliance with a treatment regimen is crucial. APIs in transdermal delivery systems are frequently attached to a skin-adhesive solid support. API of interest can also be combined with a penetration enhancer, e.g., Azone (1-dodecylazacycloheptan-2-one). Sustained release delivery systems can be inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are typically in unit dosage forms. In such form, the preparation is often subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

When it is possible that, for use in therapy, therapeutically effective amounts of API, as well as pharmaceutically acceptable salts thereof, can be administered as the raw chemical, it is possible to present the active ingredient as a pharmaceutical composition. Accordingly, the disclosure further provides pharmaceutical compositions, which include therapeutically effective mounts of API or pharmaceutically acceptable salts thereof or a prodrug thereof, and one or more pharmaceutically acceptable carriers, diluents, or excipients. When applied to a combination, the term refers to combined amounts of the active ingredients that result in the therapeutic effect, whether administered in combination, serially, or simultaneously. API and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing API, or a pharmaceutically acceptable salt thereof or a prodrug thereof, with one or more pharmaceutically acceptable carriers, diluents, or excipients.

When the compositions of this disclosure comprise a combination of API of the present disclosure and one or more additional therapeutic or prophylactic agent, both API and the additional agent are usually present at dosage levels of between about 10 to 150%, and more typically between about 10 and 80% of the dosage normally administered in a monotherapy regimen.

HDAC inhibitor and ICI can be administered together or substantially simultaneously or within a few minutes or few hours or few days or few weeks of each other.

Additional objects, advantages, and novel features of this disclosure will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Material and Methods

The following calculations and variable definitions are used:
Area under the curve (AUC^{last}=h·ng/mL).
Bioavailability (F=100×AUC^{po}×D^{iv}/AUC^{iv}×D^{po}) were F=bioavailability, AUC^{po}=dose-corrected area under curve of non-intravenous oral drug, D^{iv}=dose intravenous, AUC^{iv}=area under the curve intravenous, D^{po}=oral dose.
C^{max}=maximum serum levels.

HDAC Isoform Inhibition with SS-208: AVS100 (i.e., SS-208) is a selective histone deacetylase 6 (HDAC6) inhibitor whose chemical name is 5-(2-(3,4-dichlorobenzamido)ethyl)-N-hydroxyisoxazole-3-carboxamide. The activity of SS-208 for HDAC isoform inhibition against Class I HDACs 1 and 8, Class IIa HDACs 4, 5, 7, and 9, Class IIb HDAC6, and Class IV HDAC11 was evaluated. The HDAC profiling against Class IIa and Class IV isoforms shows that SS-208 only exhibited micromolar potency against all these isoforms whereas SS-208 demonstrated low nanomolar HDAC6 inhibitory activity with better selectivity over the other tested HDAC isoforms (Table 1).

TABLE 1
HDAC Profiles of the Compound SS-208.

| HDAC Isoform | IC50 (μM) | SI |
|---|---|---|
| HDAC1 | 1.39 ± 0.73 | 116 |
| HDAC4 | 19.5 ± 4.2 | 1625 |
| HDAC5 | 6.91 ± 0.29 | 576 |
| HDAC6 | 0.012 ± 0.002 | 1 |
| HDAC7 | 8.34 ± 0.71 | 695 |
| HDAC8 | 1.23 ± 0.59 | 103 |
| HDAC9 | 38.2 ± 5.1 | 3183 |
| HDAC11 | 5.12 ± 1.29 | 427 |

SI: Selectivity index of HDAC6 over other HDAC isoforms.

ADMET Properties of SS-208: The potential mutagenicity of SS-208 was also evaluated by incubating SS-208 with two strains of S. typhimurium (TA98 and TA1537) in the presence and absence of mammalian microsomal enzymes (S9 mix). Ames assay results showed that SS-208 did not induce ≥2-fold increases (for tester strain TA98) or ≥3-fold increases (for tester strain TA1537) in the number of colonies at any dose. This suggests that SS-208 does not possesses potential for mutagenic effects. Also, no significant inhibition was observed in the hERG assay up to 30 μM. The liver microsomal stability assay demonstrated SS-208 half-lives of 37 and 135 min in mouse and human microsomes, respectively. Furthermore, hepatocyte stability assay demonstrated half-lives of 22 and 135 min in mouse and human hepatocytes, respectively (Table 2).

TABLE 2
ADMET Profiling of Compound SS-208.

| Ames test (with and without S9) | TA98, TA1537 | negative |
|---|---|---|
| hERG test (IC50, μM) | HEK293 cells | >30 |
| Liver microsomal stability | mouse | 37 |
| (t½, min) | human | 135 |
| Hepatocytes stability | mouse | 22 |
| (t½, min) | human | 108 |

Macrophage cell culture and AVS100 treatment: Murine macrophage cell lines RAW264.7 and BMA31A7 were cultured in complete RPMI 10% FBS, 1% Pen/Stren, 1% NEAA, and glutamine. BMDMs were generated by culturing fresh bone marrow cells with 20 ng/mL of mouse-M-CSF for 4 days. On day 4 M-CSF was removed from the media, and cells were rested for 2-3 days before polarization. Polarization of murine macrophages was performed by stimulation overnight with LPS (100 ng/ml) plus IFN-γ (50 ng/ml) for M1 or with IL-4 (20 ng/ml) plus IL-13 (20 ng/ml) for M2. The human monocytic cell line THP1 was differentiated to macrophages by overnight stimulation with PMA (50 ng/ml) in RPMI 10% FBS, 1% Pen/Stren, 1% NEAA and glutamine followed by removal of PMA for 3 days before stimulation overnight with LPS (50 ng/ml) plus IFN-γ (10 ng/ml) for M1 or with IL-4 (20 ng/ml) for M2 polarization. Primary human monocytes were obtained from healthy PBMCs by ficoll gradient separation followed by magnetic monocyte enrichment (Stem Cell technologies). Human monocytes were differentiated into macrophages in human M-CSF (50 ng/ml) presence for 4 days and rested for 3 days before polarization. Macrophages were stimulated with AVS100 (10 uM) the day before polarization and re-stimulated with fresh AVS100 (10 uM) two hours before polarization. Polarization of human macrophages towards M1 was performed by incubation with IFNγ (10 ng/ml) and LPS (50 ng/ml) or for M2 with IL-4 (20 ng/ml).

Immunoblot-blot, FACs, and gPCR: For western-blot analysis cell monolayers were washed in PBS and lyzed with complete RIPA buffer containing protease, phosphatases and the HDAC inhibitor Panabinostat (10 nM). Cells were sonicated for 12 cycles at high intensity and the cell lysates were centrifuged at 12,000 rpm for 10 minutes at 4° C. to remove the cell debris. Protein quantitation on the supernatants was performed using BCA assay. Protein samples were prepared at 1 ug/uL concentration with 6× Lamelli buffer and incubated for 5 minutes at 90° C. Precast gels containing 20 μg of protein/sample were run and the gels were transferred to a PVDF membrane and stained with specific antibodies. The antibodies used for western-blot are anti-α-tubulin (mouse DM1A, Cell Signaling Tech), anti-acetyl-α-tubulin (rabbit K40, Cell Signaling Tech), anti-iNOS (rabbit polyclonal, Invitrogen), anti-Arginasel (rabbit D4E3M, Cell Signaling Tech). Anti-c-Myc (rabbit D84C12, cell Signaling Tech), anti-acetylated-c-Myc (Rabbit polyclonal Acetyl-Lys148-c-Myc, Sigma-Aldrich), anti-Stat6 (Rabbit D3H4, Cell Signaling Tech), anti-Phospho-Stat6 (Polyclonal Rabbit, Cell Signaling Tech), anti-Stat3 (Rabbit 79D7, Cell Signaling Tech), anti-Phospho-Stat3-727 (Rabbit D8C2Z, Cell Signaling Tech), anti-phospho-Stat3-705 (Rabbit D3A7, Cell Signaling Tech). Fluorescent secondary antibodies were used, donkey anti-rabbit IR Dye 800 (Licor) and goat anti-mouse IgG Azure Spectra 700 (Azure). Western-blot Images were scanned using a Licor Biosystems Imaging System.

For qPCR, RNA was isolated by Trizol purification followed by reverse transcription using iScript™ cDNA Synthesis Kit and qPCR using iQ™ SYBR® Green Supermix (Biorad). qPCR were run on real time PCR, CFX Opus 96 (Biorad). Primers used for specific amplification were as follows:

| | | |
|---|---|---|
| Tnfa: | (Qiagen, QT00104006) | |
| | (Germantown, MD, USA); | |
| Il1b: | (Qiagen, QT01048355); | |
| Arg1: | (Qiagen, PPM31770C- | |
| | 200); | |
| Fizz1: | TCCAGCTAACTATCCCTCCACTG | (SEQ ID NO: 1), |
| | T | |
| | GGCCCATCTGTTCATAGTCTTGA | (SEQ ID NO: 2); |
| Tgfb: | ACCAACTATTGCTTCAGCTTCAG | (SEQ ID NO: 3), |
| | CTCCAC | |
| | GATCCACTTCCAACCCAGGTC | (SEQ ID NO: 4); |
| Nos2: | GAGACAGGGAAGTCTGAAGCAC | (SEQ ID NO: 5), |
| | CAGCAGTAGTTGCTCCTCTTC | (SEQ ID NO: 6); |
| Gadph: | ATGGCCTTCCGTGTTCCTAC | (SEQ ID NO: 7), |
| | CAGATGCCTGCTTCACCAC | (SEQ ID NO: 8); |
| Cd64: | ATACAGGTGCCAGAGAGGTCTC | (SEQ ID NO: 9), |
| | CAGCTTATCCTTCCACGCATG | (SEQ ID NO: 10); |
| Cox2: | CGGTGAAACTCTGGCTAGACAG | (SEQ ID NO: 11), |
| | GCAAACCGTAGATGCTCAGGGA | (SEQ ID NO: 12); |
| Ccl5: | CCTGCTGCTTTGCCTACATTGC | (SEQ ID NO: 13), |
| | ACACACTTGGCGGTTCTTTCGG | (SEQ ID NO: 14); |
| Tnf: | CTCTTCTGCCTGCTGCACTTTG | (SEQ ID NO: 15), |
| | ATGGGCTACAGGCTTGTCACTC | (SEQ ID NO: 16); |
| Socs1: | TTCGCCCTTAGCGTGAAGATGG | (SEQ ID NO: 17), |
| | TAGTGCTCCAGCAGCTCGAAGA | (SEQ ID NO: 18); |
| Ido: | GCCTGATCTCATAGAGTCTGGC | (SEQ ID NO: 19), |
| | TGCATCCCAGAACTAGACGTGC | (SEQ ID NO: 20); |
| Cd200r1: | CGTCTCCCATTTGACTGGCAAC | (SEQ ID NO: 21), |
| | CCAAATGAATCCCACGATGGTCA | (SEQ ID NO: 22); |
| Mrc1: | GGGTTGCTATCACTCTCTATGC | (SEQ ID NO: 23), |
| | TTTCTTGTCTGTTGCCGTAGTT | (SEQ ID NO: 24); |
| Tgm2: | TGTGGCACCAAGTACCTGCTCA | (SEQ ID NO: 25), |
| | GCACCTTGATGAGGTTGGACTC | (SEQ ID NO: 26); |
| Il10: | GACTTTAAGGGTTACCTGGGTTG | (SEQ ID NO: 27), |
| | TCACATGCGCCTTGATGTCTG | (SEQ ID NO: 28). |

For flowcytometry single cell suspensions were prepared in FACs buffer (PBS 1% FBS) and staining was performed for 20 min at 4° C. before acquisition in BD Fortessa cytometer. The following antibodies from Biolegend were used: AF700 anti-mouse CD3 (17A2); BV421 anti-mouse/human CD11b (M1/70); APC/Fire750 anti-mouse CD45.2; APC-anti-mouse CD80; PE/Cyanine7 anti-mouse CD206 (MMR); BV785 anti-mouse F4/80 (BM8); BV605 anti-mouse Ly-6C (HK1.4); BV650 anti-mouse CD4 (GK1.5); APC/Fire750 anti-mouse CD8a (53-6.7); PB anti-mouse CD25 (PC61); BV605 anti-mouse/human CD44 (IM7); BV785 anti-mouse CD45.2 (104); PE anti-mouse CD62L (MEL-14). Live dead exclusion was done by using LIVE/DEAD™ Fixable Aqua Dead Cell Stain Kit, (Thermofisher). CD45⁺ cell sorting was performed using a BD Facsaria cell sorter (BD biosciences).

RNAseq and scRNAseq: For the scRNAseq analysis, sorted CD45+ cells were verified for a viability above 95% before being subjected to a 10× Genomics workflow. RNAseq libraries were prepared following the manufacturer's instructions. The feature-barcode matrices generated by the 10× Genomics CellRanger pipeline were analyzed with an SCTransform workflow (Seurat v4.3.0.1) in RStudio. Cells with greater than 10% mitochondrial counts, fewer than 100 genes detected, or with a number of counts greater than the 93rd percentile of counts were removed. Cell type recognition was performed using the reference-based scRNA-seq annotation package, SingleR. The Immunological Genome Project (ImmGen) was used as the reference dataset. The Seurat object was filtered to include the major immune cell populations present.

To perform the macrophage analysis, dimensional reduction analysis was run on a subset of the original Seurat object that only contained cells identified as macrophages by the SIngleR celltype annotation analysis. The differential gene expression functionality of the Seurat package was used to examine differences in gene expression profiles between the macrophage subclusters and to infer the TAM subcluster functional classifications. To perform the T cell analysis, a subset of the original Seurat object was created using the CellSelector function to manually select cells that belonged to clusters representing T-cells as determined by gene expression and/or SingleR analysis. The differential gene expression functionality was used to determine genes that were differentially expressed in T cells between samples. A select number of these genes are represented in the heatmaps and histograms. Functional analysis of gene sets was performed using the online tool Enrichr (Curr Protoc., 2021, 1, e90).

In vivo tumor growth: Analysis of tumor growth using the murine SM1 melanoma model were performed in accordance with IACUC guidelines at Georgetown University. Murine melanoma tumors were established by implanting 0.75×10⁶ SM1 cells in the right flank area of 6-8 weeks-old female C57BL/6 mice. When the tumors were palpable (5×5 mm), mice were randomized into treatment groups, and AVS100 and vehicle control were administered by oral gavage daily in a suspension containing PEG-6000, Microcrystalline Cellulose, Corn Starch, Silicon Dioxide, Magnesium Stearate and Sterile water. Anti-PD1 or isotype control (BE0146-CUST and BE0089-CUST, Bio X cell) were injected i.p. (15 mg/kg) in 100 ul PBS every other day. Tumor growth was monitored using electronic calipers, the length and width of detected tumors were measured every other day and tumor volumes calculated using the formula ½*length*(width)². The body weights of animals were recorded once a week using a portable scale to monitor general health.

For the CT26 colon cancer model animal studies were performed by Adgyl Lifesciences. Mice were implanted with 0.5×106 CT26 cells in the right flanks of 7-8 week-old female Balb/c mice. 8 days after implantation, mice were gavaged daily with 100 mg/kg AVS100 in PEG-6000 suspension (vehicle containing 5% Ethanol+PEG6000 (2.4× of AVS100) in sterile water) and treated with anti-PD1 10 mg/kg i.p. every 4 days. Tumor volume and body weight were monitored twice weekly.

Tumor dissection and single-cell suspension: Tumor single-cell suspensions were obtained by mincing the tumor tissue with scalps in petri dishes with complete media, followed by incubation of the minced tumors in a digestion solution (collagenase I, collagenase IV, hyaluronidase V, and DNase I) at 37° C. with rotation for 30-45 min. The digested solution was passed by a cell strainer to remove undigested material, centrifuged, washed and resuspended in complete media.

TCRseg and clonotypic analysis: Sorted intratumoral CD45⁺ cells and spleen CD8⁺CD44⁺ T-cells were resuspended in Trizol for mRNA extraction and sent to perform TCRseq and clonotype analysis by Genewiz/Azenta using their streamlined TCR sequencing platform and bioinformatics analysis. The mapping and quantification of the T-cell repertoire was provided by the Azenta bioinformatics pipeline, which mapped the reads to the appropriate V(D)J genes. The clones that were identified were ranked (in decreasing order) by frequency, and the Pielou index was calculated for each condition and replicate as follows:

$$Pielou Index = \frac{- {\sum }_{i} {p}_{i} log ( {p}_{i} )}{log ( N )}$$

where pi is the proportion of the ith clone and N is the total number of clones. The pie chart was generated by identifying the top 5 most abundant clones and generating a respectively sized plot. Clone counts are represented from the 10 most abundant T-cell clones.

LS-MS: Plasma samples were obtained using EDTA as anticoagulant and run by Adgyl Life Sciences using a fit for purpose LC-MS/MS method. Briefly, plasma samples were run in a triple Quad 6500+LC-MS/MS System using a 50:50 (ACN:Water v/v) working solution diluent. A mobile Phase A: 2 mM Ammonium formate with 0.1% Formic acid, a mobile Phase B: Acetonitrile with 0.1% formic Acid Isocratic mode; Pump A:B: 50:50. Using a Phenomenex Phenyl-Hexyl column of Sum particle size. To identify and quantitate AVS100 (SS208) peaks, chromatograms were acquired by using Analyst® software version 1.7.3 Analyst data was exported to Watson LIMS software version 7.5 to determine calculated concentration of calibration curve standards, quality control and back calculated concentration of unknown study samples. The calibration curve was generated using area ratio of analyte area: area of internal standard vs concentration and was regressed using linear regression equation and weighting of 1/x2.

$$Y = mX + C$$

Where, Y=peak area ratio of analyte over internal standard, C=Intercept, X=Concentration of analyte, m=Slope

The plasma pharmacokinetic parameters of SS-208 were determined from concentration time curves obtained following a single IV and oral route of administration in male C57BL/6 mice. Values are expressed as mean and n=3 mice/time point/group.

### Discussion

Inhibition of HDAC6 has been associated with an increased proinflammatory tumor microenvironment and antitumoral immune responses. Experiment below show that the HDAC6 selective inhibitor, e.g., AVS100, increased acetyl-cMyc, reduced total c-Myc levels and inhibited M2 polarization in murine and human macrophages. Oral administration of AVS100 had an antitumoral effect in SM1 melanoma and CT26 colon cancer models and increased the efficacy of anti-PD1 treatment, leading to complete remission in melanoma and increased response in colon cancer. Treatment led to an increase in proinflammatory tumor infiltrating macrophages (TAMs), an increase of intratumoral CD8 effector T-cells and an inflammatory and T-cell effector gene signature. Acquired T-cell immunity and long-term protection were evidenced as increased immunodominant T-cell clones after AVS100 treatment. Furthermore, AVS100 showed no mutagenicity, toxicity, or adverse effects in preclinical good laboratory practice (GLP) studies.

To date, five HDAC inhibitors are clinically available: Vorinostat, Romidepsin, Panobinostat, Belinostat, and Tucidinostat for the treatment of cutaneous T-cell lymphoma (CTCL), multiple myeloma, and peripheral T-cell lymphoma (PTCL). They target multiple HDAC isoforms, and their therapeutic efficacy is related to broad chromatin changes and direct toxicity to cancer cells. Due to their broad mechanism of action, they have high toxicity profiles, limiting their clinical application to aggressive hematological malignancies. Approximately thirty HDAC inhibitors, including HDAC6-specific inhibitors, have been evaluated in clinical trials, mainly targeting hematologic neoplasms but also for other indications such as solid cancers, autoimmune diseases, and neurological disorders.

HDAC6 differs from other HDAC proteins in that it localizes largely to the cytoplasm with the capacity to deacetylate a variety of non-histone proteins. The first characterized target of HDAC6 was α-tubulin, leading to the regulation of microtubules, cytoskeleton, intracellular trafficking, and cellular responses to stress. Heat shock protein 90 (HSP90) is another known target of HDAC6 controlling the regulation of diverse cell signals and protein stability. HDAC6 also controls cell migration by direct acetylation of cortactin and actin binding, and it deacetylates Ku70 and Survivin, suppressing apoptosis, among other functions.

HDAC6 has been shown to regulate both proinflammatory and anti-inflammatory functions in macrophages. However, different functions of HDAC6 are related to the specificity of the small molecule inhibitors, which may also inhibit other HDACs. The use of highly specific inhibitors of HDAC6 identified that HDAC6 signals lead to an anti-inflammatory phenotype in tumor-associated macrophages (TAM), and its inhibition to an increased proinflammatory tumor microenvironment and an antitumoral immune response in colon cancer, breast cancer and melanoma models. A specific blockade of M2 macrophage polarization by pharmacological HDAC6 inhibition has recently been described.

Inhibition of HDAC6 by small molecule inhibitors has shown promise as an immunotherapeutic agent in preclinical tumor models. However, as measured in the AMES test, most HDAC6 inhibitors contain a hydroxamate group and have mutagenesis potential, precluding their wide clinical application. A few exceptions to this belong to the HDAC6 inhibitors AVS100 (SS-208) and SW100, which are AMES-negative despite containing a hydroxamate group and are suitable for clinical translation. Based on the non-mutagenic properties of AVS100 and high HDAC6 specificity, the present inventors have theorized that such HDAC6-selective compounds would be useful antitumoral compounds, in particular for solid tumors.

### Results

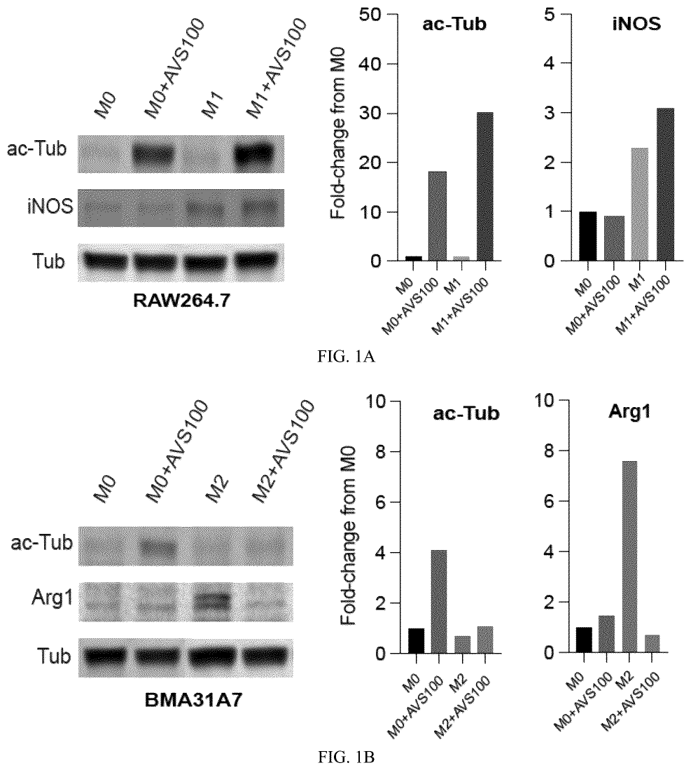

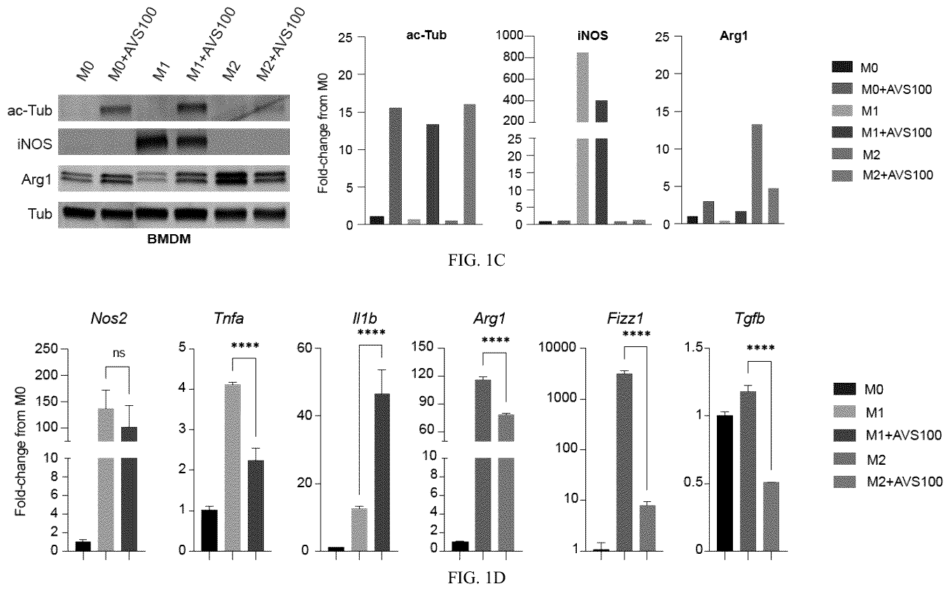

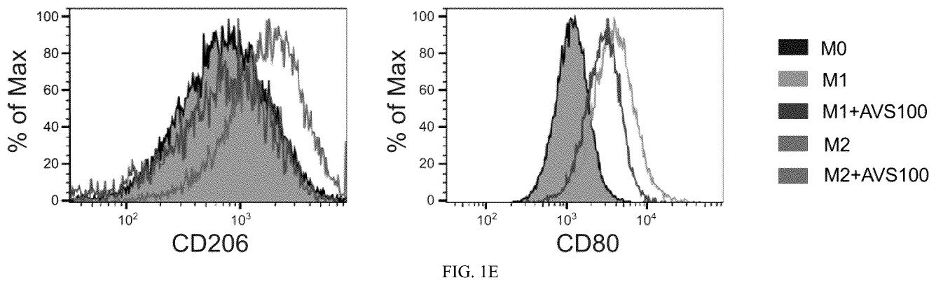

AVS100 inhibits M2 polarization of murine macrophages with negligible effects on M polarization: To better understand how AVS100 directly affects macrophage phenotype and function, the murine cell line Raw264.7 was polarized towards the proinflammatory M1 phenotype by treatment with LPS plus IFNγ and the cell line BMA31A7 towards the anti-inflammatory M2 phenotype by treatment with IL-4 plus IL-13. Two different cell lines were used as they are better models for polarization towards M1 and M2, respectively. Acetylated α-tubulin levels was measured as a readout of AVS100 HDAC6 inhibitory activity and inducible nitric oxide synthase (iNOS) and Arginase 1 (Arg1) levels as markers of M1 and M2 polarization, respectively. AVS100 treatment increased acetyl-α-tubulin levels in both models. It blocked Arg1 upregulation under M2 polarizing conditions, but it did not affect the increase of iNOS levels under M1 polarizing conditions (FIGS. 1A and 1), suggesting a specific inhibition of M2 but not M1 polarization in murine macrophages.

The M2 inhibitory function of AVS100 was also observed in primary cell cultures of murine bone marrow-derived macrophages (BMDM), as reduced Arg1 levels in M2 polarizing conditions. A partial reduction of iNOS levels was also observed after AVS100 treatment under M1 polarizing conditions (FIG. 1C). However, qPCR analysis of BMDM showed that AVS100 did not affect Nos2 levels. Additionally, increased Il1b levels in M1 cells and inhibition of the M2 markers Arg1, Fizz1, and Tgfb in M2 cells were observed (FIG. 1D). A preferential activity of AVS100 during M2 polarization was also observed as a complete blockade in the generation of CD206^{hi}F4/80⁺CD11b⁺ M2 macrophages, with normal generation of CD80^{hi}F4/80⁺CD11b⁺ M1 macrophages from BMDM (FIG. 1E).

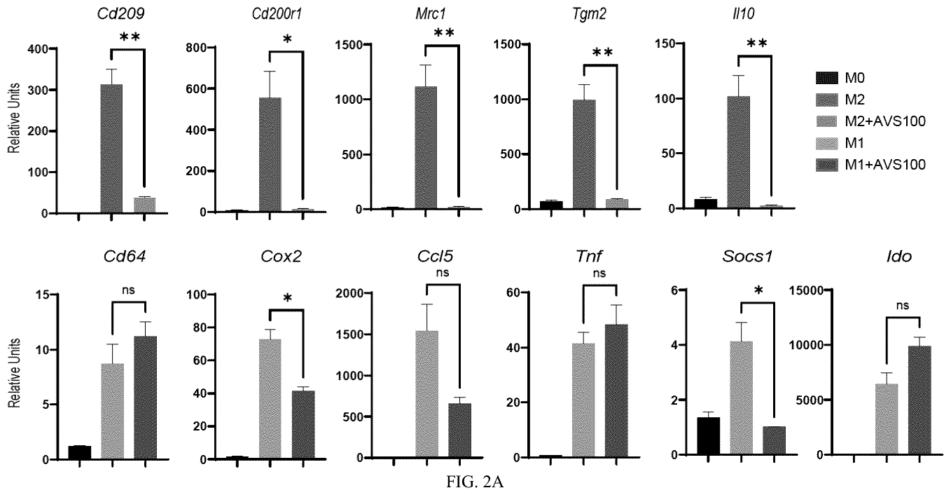

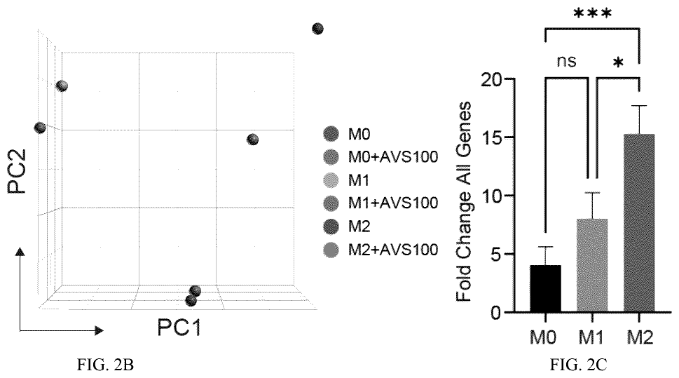

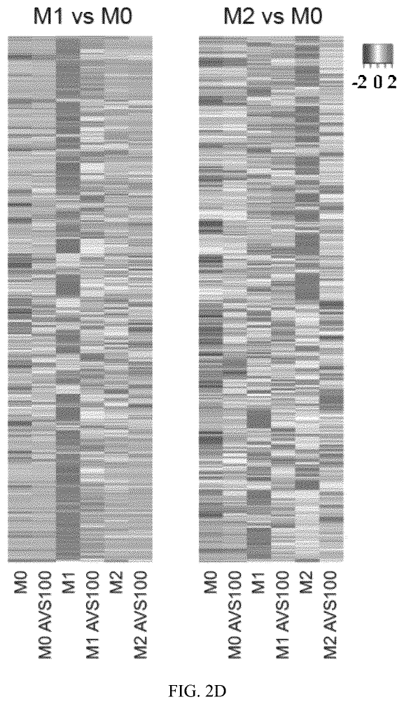

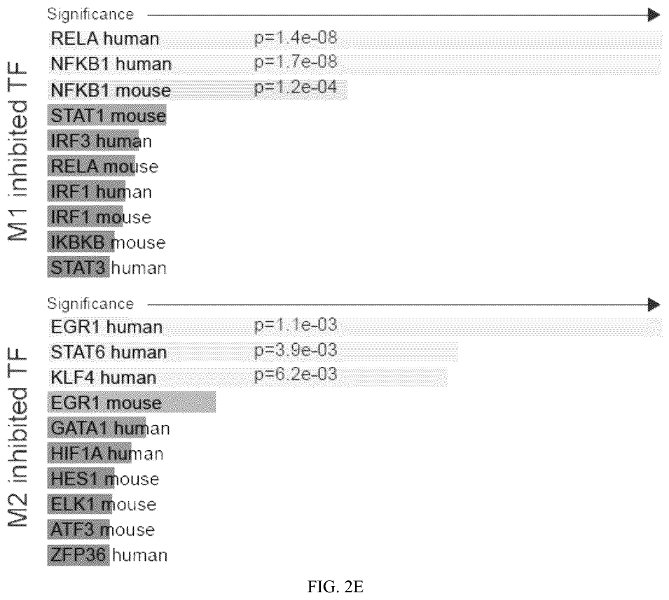

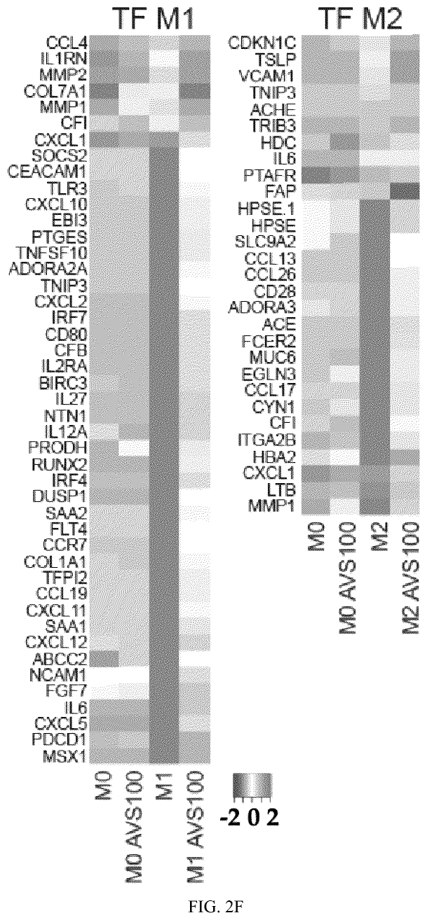

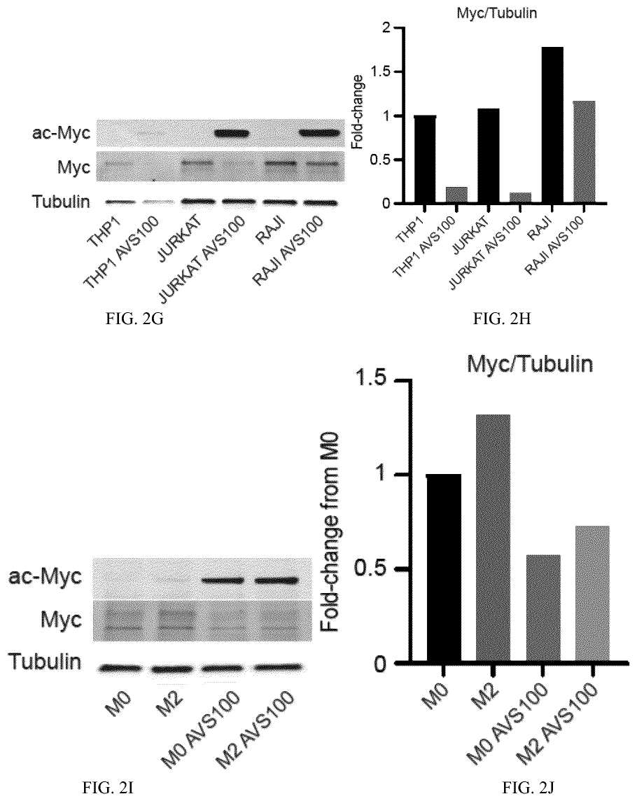

AVS100 preferentially blocks the anti-inflammatory transcriptional program in human macrophages: To investigate the effect of AVS100 in human macrophages, macrophages were differentiated from PBMCs in the presence or absence of AVS100. As a readout of polarization, changes were analyzed in the expression levels of genes associated with the M1 and M2 phenotype in human macrophages. It was observed that AVS100 treatment prevented the induction of the M2 markers Cd209, Cd200r1, Mrc1, Tgm2, and Il10. AVS100 did not affect M1 markers Cd64, Tnf and Ido induction and partially inhibited Cox2, Ccl5, and Socs1 induction (FIG. 2A).

To better understand the mechanisms of AVS100 effects, RNAseq analysis of primary human macrophages not stimulated with cytokines and unpolarized (M0) or polarized towards M1 and M2 phenotypes was performed in the presence or absence of AVS100. Principal complement analysis (PCA) showed that all treatment groups preferentially clustered based on the polarizing conditions (M0, M1, and M2) and not on AVS100 treatment (FIG. 2B), indicating that AVS100 treatment had local effects within each cluster and did not affect macrophage identity. K-mean clustering analysis of the top 2000 genes differentially expressed across all samples identified three clusters corresponding to M0, M1, and M2 polarizing conditions. See, Table below. In the table below, cluster A corresponded to genes upregulated in M1 with enriched biological classes, including Interferon and cytokine signaling; cluster B consisted of genes upregulated in M0 samples enriched for GO terms related to cell cycle and metabolism; and cluster C comprised genes upregulated in M2 samples, with enriched GO terms related to immunoregulatory interactions between lymphoid and non-lymphoid cells as well as IL4, IL10, and IL13 signaling.

Pathways

Cluster A N = 1081

| | |
|---|---|
| A | Interferon Signaling |
| A | Cytokine Signaling in immune system |
| A | Immune System |
| A | Interferon gamma signaling |
| A | Interferon alpha/beta signaling |
| A | Response to metal ions |
| A | Metallothioneins bind metals |
| A | Signaling by Interleukins |
| A | Innate Immune System |
| A | GPCR ligand binding |
| A | Nucleotide binding domain leucine rich repeat |
| | containing receptor NLR signaling pathways |
| A | Post-translational protein phosphorylation |
| A | DDX58/IFIH1-medlated Induction of Interferon- |
| | alpha/beta |
| A | Amino acid transport across the plasma membrane |
| A | Class A/1 Rhodopsin-like receptors |

Cluster B: N = 447

| | |
|---|---|
| B | Cell Cycle Mitotic |
| B | Cholesterol biosynthesis |
| B | Resolution of Sister Chromatid Cohesion Cell |
| | Cycle Checkpoints |
| B | RHO GTPases Activate Formins |
| B | Mitotic Prometaphase |
| B | Amplification of signal from the kinetochores |
| B | Amplification of signal from unattached |
| | kinetochores via a MADZ inhibitory signal |
| B | G1/S-Specific Transcription |
| B | Mitotic Spindle Checkpoint Metabolism of lipids |
| B | Metabolism |
| B | RHO GTPase Effectors |
| B | Mitotic G1-91/S phases |
| B | Signaling by Rho GTPases |

Cluster C: N = 472

| | |
|---|---|
| C | Immune System |
| C | Immunoregulatory interactions between a |
| | Lymphoid and a non-Lymphoid cell |
| C | Interleukin-4 and Interleukin-13 signaling |
| C | Signaling by Interleukins |
| C | GPCR ligand binding |
| C | Interlenkin-10 signaling |
| C | Class A/1 Rhodopsin like receptors |
| C | Innate Immune System |
| C | Cytokine Signaling in Immune System |
| C | Hemostasis |
| C | Platelet degranulation |

To interrogate if there was a more pronounced effect of AVS100 within the different polarizing conditions, the average fold change of all genes in M0, M1, and M2 by AVS100 treatment was analyzed using a formula that provides a positive value by AVS100-mediated inhibition of gene expression. Interestingly, AVS100 had a global inhibitory effect in all conditions, but inhibition of transcription was significantly higher in M2 cells (FIG. 2C). This effect is depicted in the PCA analysis of all groups, with a higher distance between M2 and M2 AVS100 than between M1 and M1AVS100 or M0 and MOAVS100 conditions (FIG. 2B). Despite the more substantial effect of AVS100 in M2 cells, an inhibitory effect was also observed on selected genes that were upregulated in M1 or M2 polarizing conditions (FIG. 2D).

Using the Trust transcription factor pathway database, on list of genes upregulated in M1 and M2 conditions that AVS100 also downregulated, it was identified that AVS100 significantly inhibited genes belonging to the RelA/NFKB program in M1 cells while it preferentially inhibited genes belonging to EGR1, STAT6, and KLR4 program in M2 cells, pathways that control M1 and M2 polarization in macrophages respectively (FIG. 2E). Forty-five (45) genes that belong to the RelA/NFKB transcriptional program was identified as being inhibited by AVS100 in M1 cells (FIG. 2F). Interestingly, 21 of these genes have anti-inflammatory functions and are involved in the negative feedback control of inflammation. Among these genes is Dusp1 that regulates a subset of LPS-induced genes involved in controlling inflammation, protecting mice from lethal endotoxin shock; Il1rn, which blocks inflammatory IL-1 signaling; Ebi3, a subunit of IL35 with anti-inflammatory functions; Tnfsf10 (Trail) which enhances neutrophil apoptosis to resolve inflammation; Cecam1, Irf4, and Tnip3 that inhibit the production of proinflammatory cytokines by macrophages and NFKB functions. Other genes with anti-inflammatory functions that showed downregulation by AVS100 in M1 conditions are Prodh, Cfb, Cxcl11, fg7, Il27, Socs2, Il2ra, Saa1, Il6, Adora2a, Pdcd1, Cxcl12, Col1a1 (FIG. 2F). Genes corresponding to the EGR1, STAT6, and KLF4 programs that were upregulated during M2 polarization and showed downregulation by AVS100 include Itga2b, involved in phagocytosis of apoptotic cells, Adora3 and Ifitm3, which block proinflammatory responses, among others associated with M2 macrophages.

Altogether, AVS100 treatment preferentially inhibits M2 polarization in mouse and human macrophages and partially inhibits genes upregulated during M1 polarization.

AVS100 increases c-Myc acetylation and reduces cMyc levels: Several signaling pathways, including MAPK, Akt and STAT6 have been identified to regulate M2 polarization in macrophages. STAT6 phosphorylation occurs downstream of IL-4 signals and initiates the M2 program. STAT3 activation occurs downstream of IL-6 and IL-10 and plays a role in M2 polarization of macrophages in vivo. HDAC6 inhibition was shown to inhibit STAT3 phosphorylation under IL-6 stimulation in cell lines.

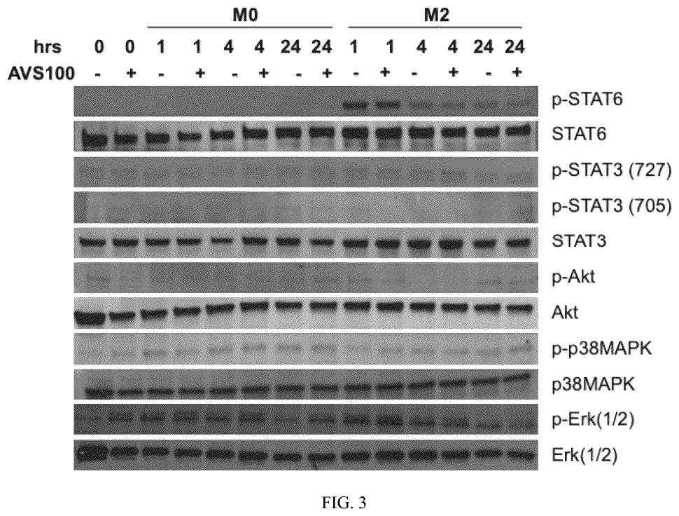

Evaluation was conducted to see if phosphorylation of STAT6, STAT3, Akt and MAPK may occur under M2 polarizing conditions in vitro and if AVS100 would have an inhibitory effect. The THP1 human monocytic cell line was used as a model system, which was differentiated into macrophages. Results showed that, as expected, STAT6 was phosphorylated in response to IL-4. However, this phosphorylation was not inhibited by AVS100 treatment. In addition, no significant increase in STAT3 phosphorylation at Tyr705 or Ser727, Akt, Erk or p38 was detected after IL-4 treatment or regulation by AVS100, suggesting that these pathways were not responsible for the AVS100 effects in inhibiting the IL-4-mediated M2 polarization (FIG. 3).

c-Myc induction occurs downstream of IL-4 signals, which is essential for the M2 polarization of human macrophages, and HDAC6 inhibition was recently shown to reduce c-Myc protein levels in B-cell lymphoma. Therefore, it was determined whether AVS100 may increase the acetylation of c-Myc and reduce total c-Myc protein levels as a mechanism inhibiting M2 polarization. AVS100 treatment led to increased acetyl-c-Myc and reduced total c-Myc levels in myeloid and lymphoid lymphoma cell lines (FIGS. 2G and 2H). AVS100 also reduced c-Myc levels in M0 and M2 polarized macrophages (FIGS. 2I and 2J), suggesting a potential mechanism for AVS100 effects in macrophages.

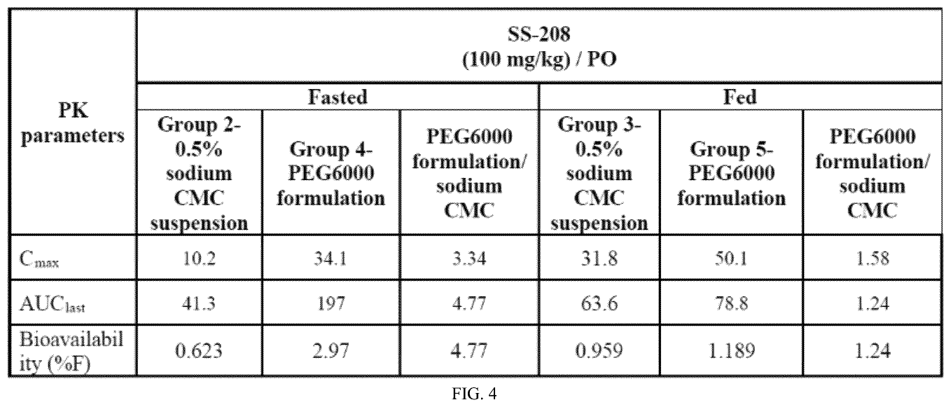

AVS100 anti-PD1 combination therapy led to tumor remission in a melanoma model: The HDAC6 inhibitor Nexturastat-A has an antitumoral effect and potentiates anti-PD1 therapy in melanoma tumor models. However, tumors are not completely eliminated. The present inventors have previously found that AVS100 reduces tumor growth as a standalone therapy. In this study, AVS100 was evaluated to determine whether it would also potentiate the antitumoral effects of anti-PD1 treatment. Although previous studies used intraperitoneal (i.p.) administration of HDAC6 inhibitors, in this study AVS100 was administered by oral gavage to better correlate findings to preclinical efficacy. To establish the optimal conditions for AVS100 administration, the bioavailability of AVS100 after oral gavage was first identified using two different formulations and measured AVS100 levels in blood by LS-MS. For oral gavage, CMC or PEG6000 formulations were used in fed or starved mice. Data showed that AVS100 administered with a PEG6000 formulation led to higher maximum concentrations in blood (C^{max}), higher area under the curve (AUC) levels, and bioavailability compared to the CMC formulation as shown in FIG. 4. Based on these results, PEG6000 was selected for all subsequent in vivo experiments.

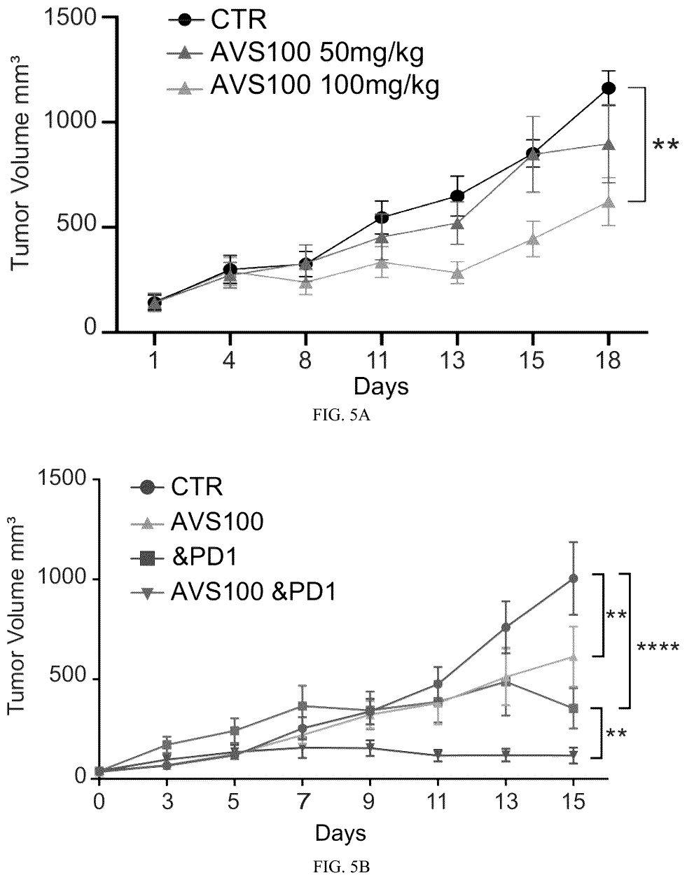

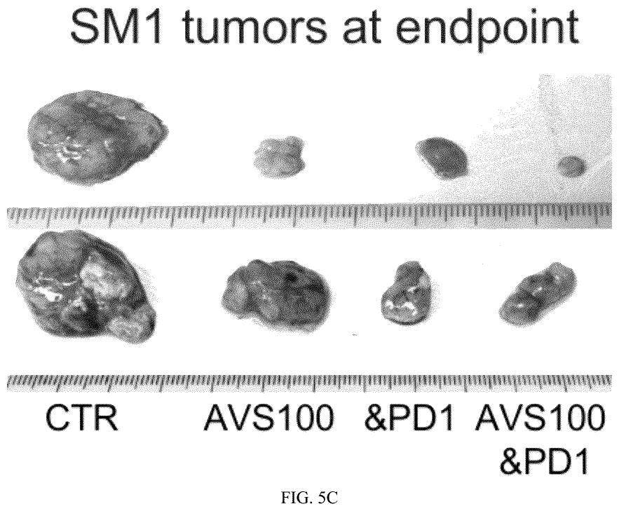

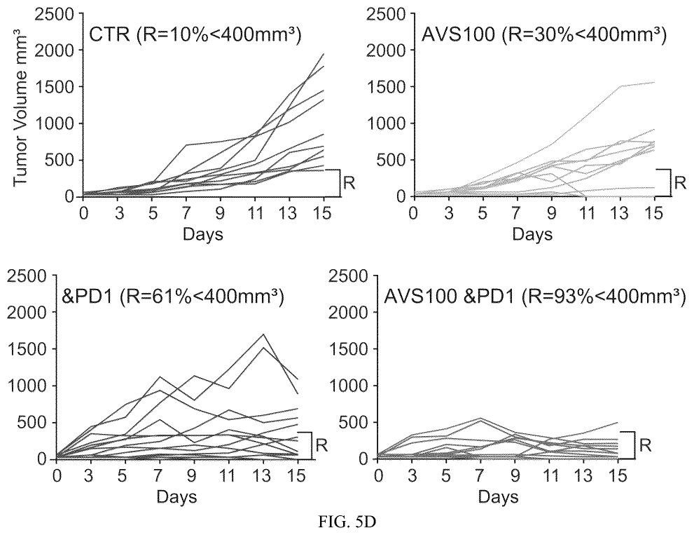

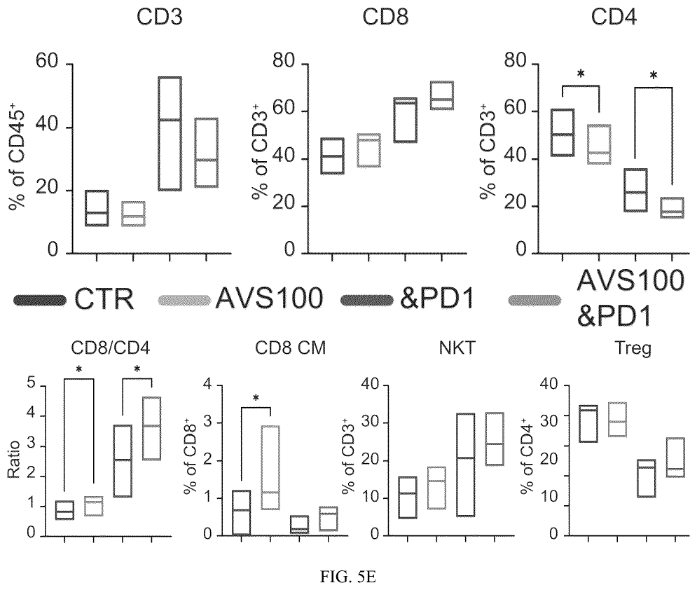

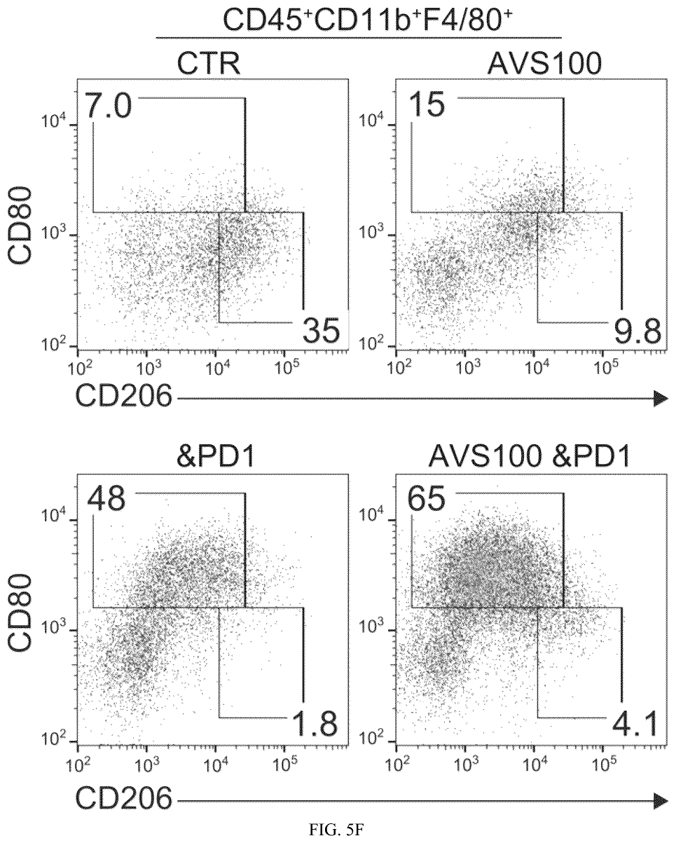

The standalone effect of AVS100 was tested on melanoma tumor growth. SM1 cells were injected subcutaneously (s.c.). Once tumors were palpable, daily oral gavage was started with AVS100 or vehicle. AVS100 at a dose of 100 mg/kg significantly reduced tumor growth (FIG. 5A). The effect of daily oral gavage with AVS100 in combination with anti-PD1 was then tested. AVS100 and anti-PD1 treatment as standalone therapies similarly reduced tumor growth. Combination therapy with AVS100 plus anti-PD1 led to a significant reduction of tumor growth compared to anti-PD1 treatment alone (FIG. 5B). Importantly, combination therapy led to complete remission that was not achieved by anti-PD1 treatment alone. Representative tumor images show the size of dissected tumors from different treatment groups (FIG. 5C). Analysis of tumor growth in individual mice identified that 61% of tumors were responders in the anti-PD1 treatment group with tumor volume lower than 400 mm³ at the endpoint and 6/13 mice in complete remission, while AVS100 plus anti-PD1 combination therapy led to 93% of responder mice with 10/13 mice in complete remission (FIG. 5D).

AVS100 treatment leads to an increase in intratumoral CD8 T-cells and an increased M1/M2 ratio of tumor-associated macrophages (TAM) in melanoma tumors: Flow cytometry analysis of intratumoral lymphocytes identified that anti-PD1 treatment led to increased T-cell infiltrates corresponding to CD8 T-cells, a proportional reduction of CD4 T-cells, and decreased T-reg infiltration. AVS100 treatment also led to a reduction of intratumoral CD4 T-cells and an increase of CD8 T-cells. Interestingly, the effect of anti-PD1 and AVS100 on the intratumoral T-cell compartment were additive as AVS100 treatment led to a significant increase of CD8/CD4 ratio in control as well as in anti-PD1 groups (FIG. 5E, from left to right: CTR; AVS100; &PD1; AVS100&PD1). AVS100 standalone treatment led to a specific increase in central memory CD8 T-cells characterized as CD62L⁺CD44⁺.

TAMs was identified as CD45⁺CD11b⁺F4/80⁺ cells and classified them within the M1 or M2 subsets based on surface levels of CD80 and CD206, respectively. Although classification within these groups is arbitrary, as most TAMs have a mixed phenotype, it serves as a framework to identify general macrophage phenotype changes related to the treatment. AVS100 treatment led to an increase in CD80 levels and a reduction in CD206 levels compared to the control group (FIG. 5F), which was observed as a significant increase in the M1/M2 ratio. (FIG. 5G). The effect of anti-PD1 treatment was more pronounced, observed as a more robust upregulation of CD80 and downregulation of CD206 and a higher increase of M1/M2 ratio (FIGS. 5F and 5G). The substantial increase of M1-like and reduction of M2-like macrophages by anti-PD1 treatment could not be further increased by AVS100 plus anti-PD1 combination treatment.

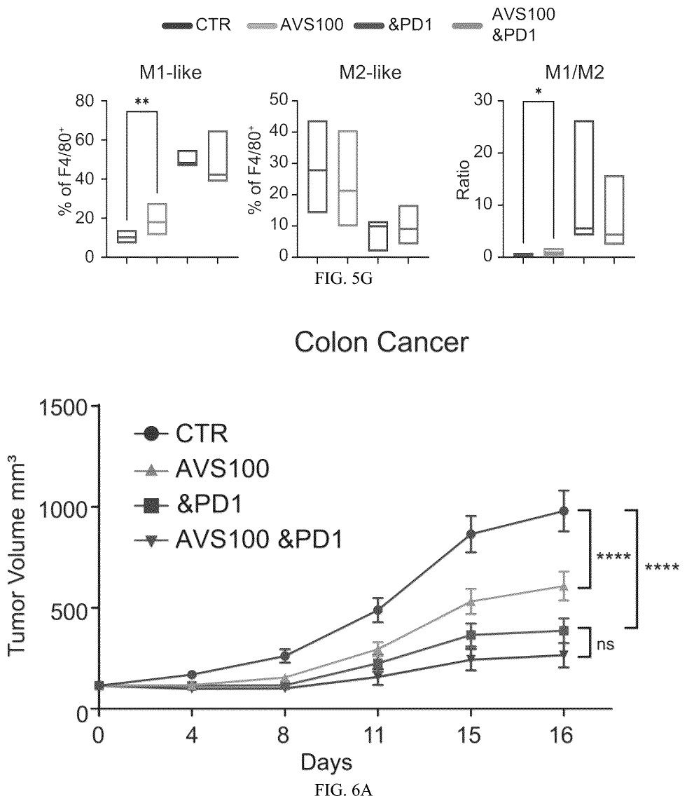

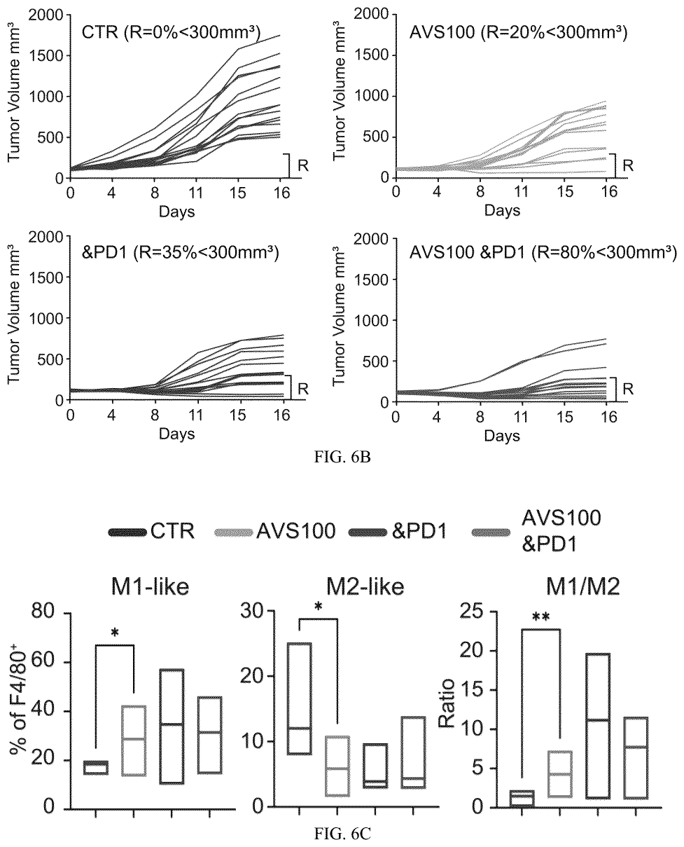

AVS100 has a standalone antitumoral effect and improves anti-PD1 therapy in a colon cancer model: To test if AVS100 effects would benefit other tumor models, its effects was evaluated as a standalone therapy and combined with anti-PD1 in the CT26 colon cancer model. CT26 is a model highly responsive to immunotherapy and a standard model to evaluate antitumoral effects. AVS100 had a standalone antitumoral response with significant tumor growth inhibition (FIG. 6A). However, tumors were not eradicated, and 20% of mice were responders with tumor volume lower than 300 mm³ compared to 0% in the control group (FIG. 6B). Anti-PD1 treatment reduced tumor growth in 35% of responders (FIGS. 6A and 6B). AVS100 plus anti-PD1 combination therapy showed an increase in responders from 35% in anti-PD1 treatment alone to 80% in AVS100 plus anti-PD1 combination therapy, suggesting improved efficacy (FIG. 6B). Similar to the direct effects of AVS100 in increasing the M1/M2 TAM ratio in melanoma tumors, AVS100 treatment alone led to a significant increase of M1-like and a significant reduction of M2-like TAMs (FIG. 6C). Interestingly, the M1/M2 ratio increase observed by anti-PD1 treatment could not be further enhanced by AVS100 plus anti-PD1 combination treatment, a similar effect to that observed in melanoma tumors.

Data shows that AVS100 treatment increases M1/M2 TAMs in melanoma and colon cancer tumor models. These changes were associated with a standalone antitumoral effect and with the improvement of anti-PD1 therapy, leading to complete tumor remission in melanoma and enhanced response in colon cancer.

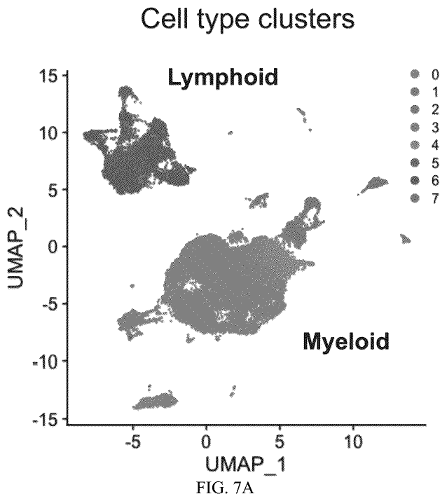

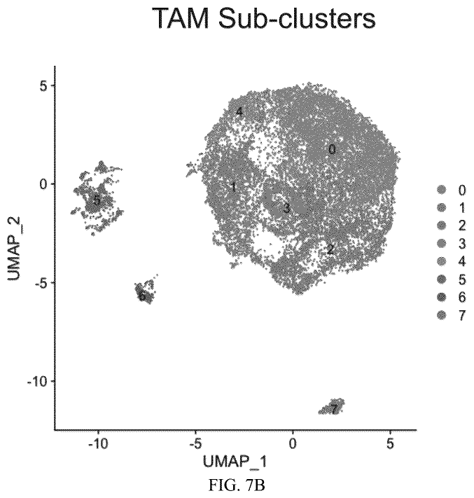

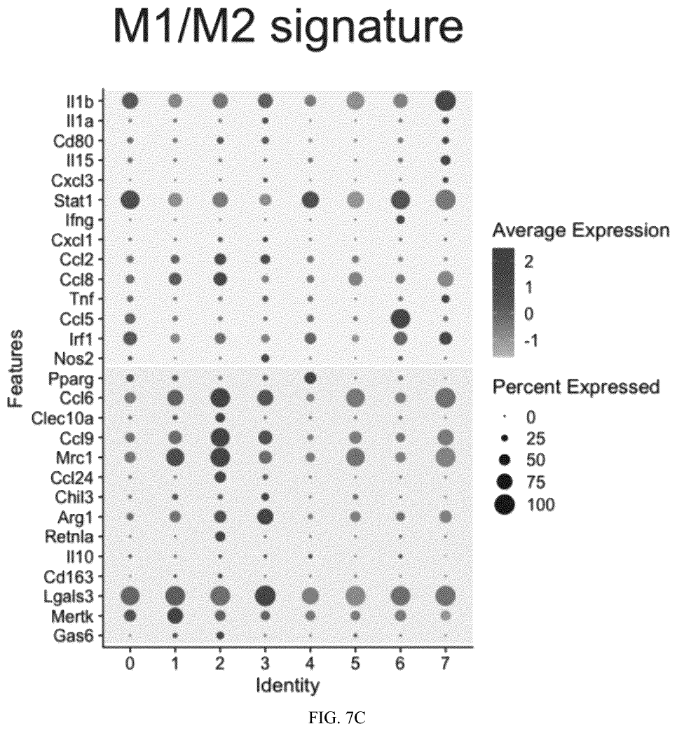

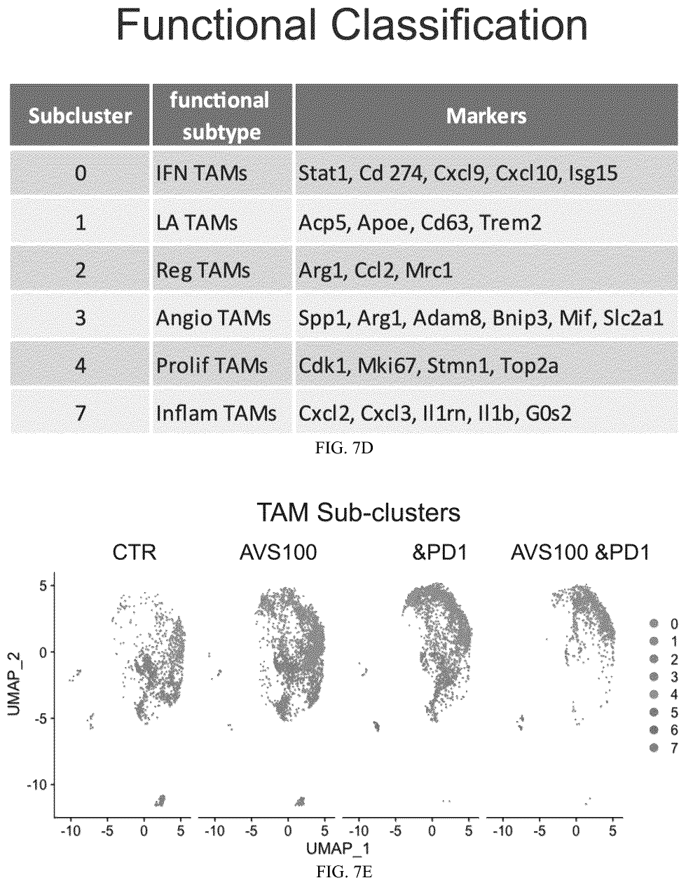

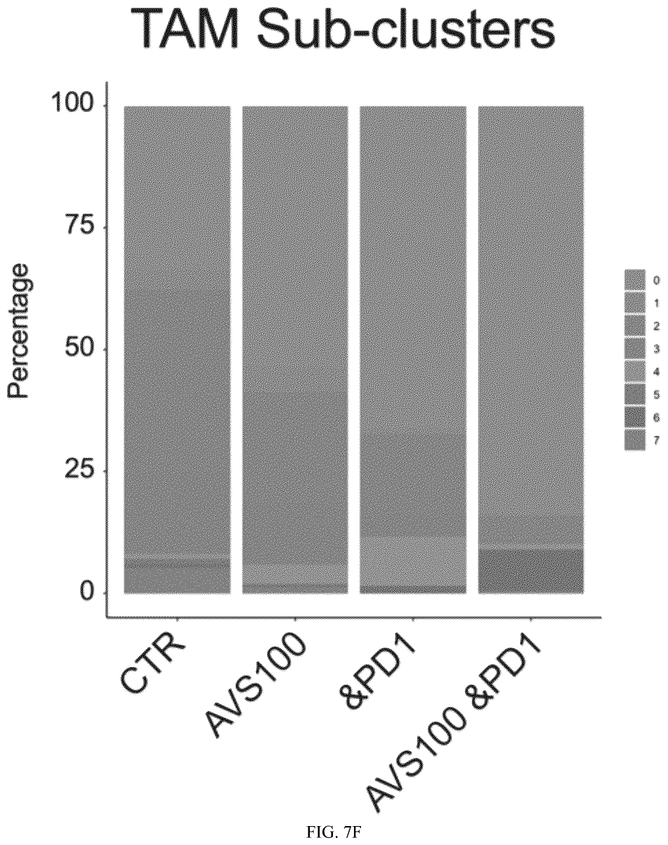

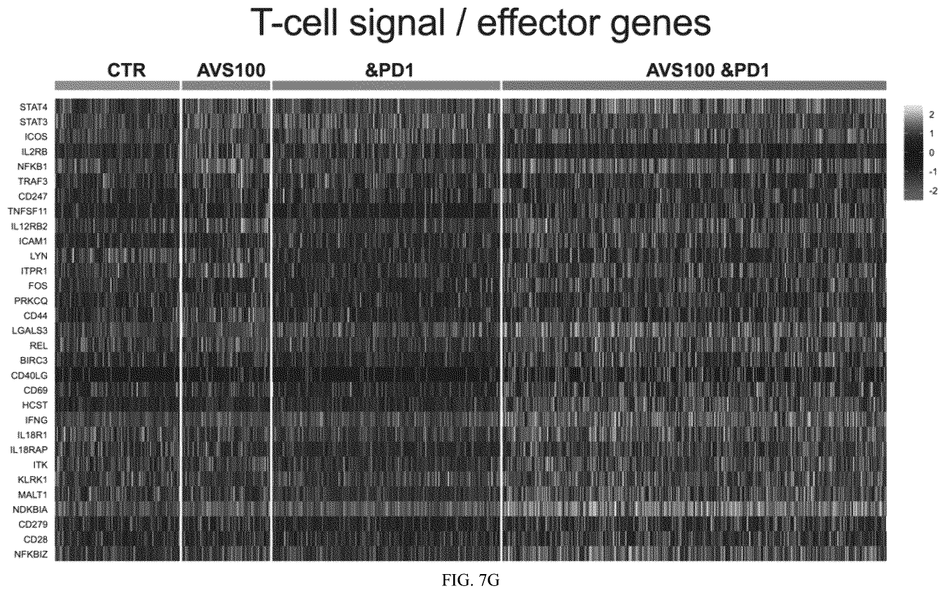

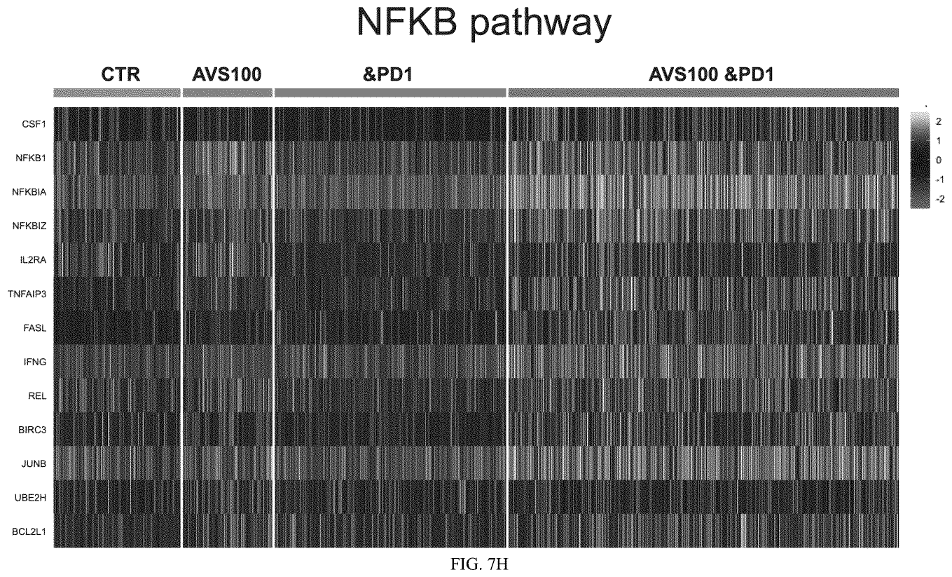

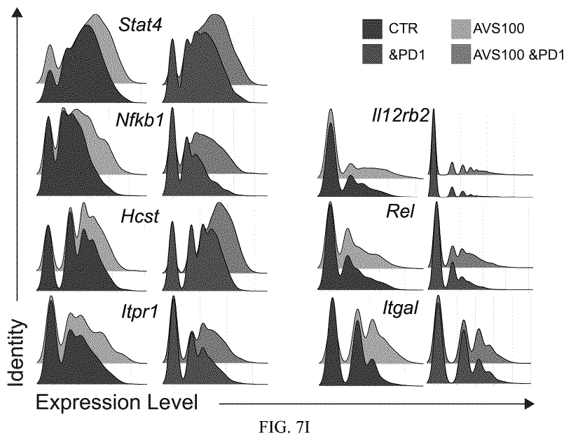

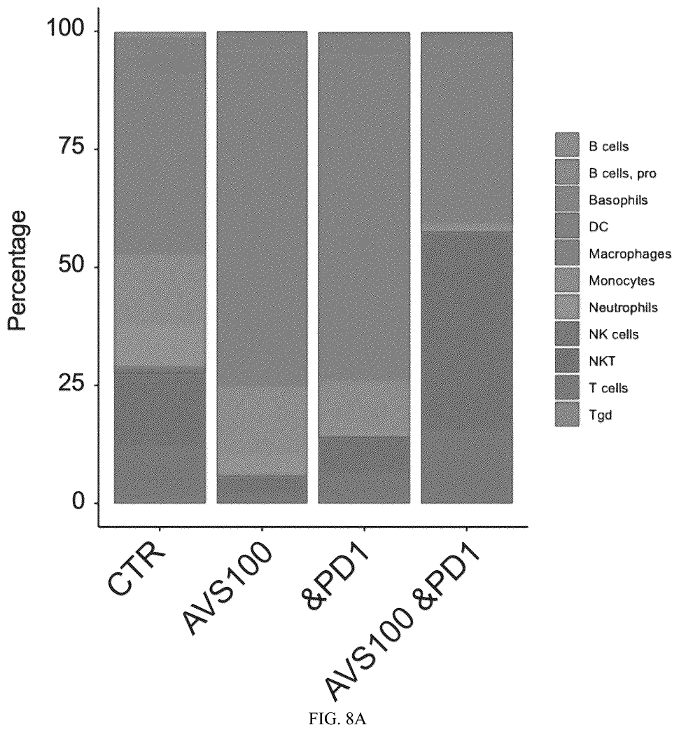

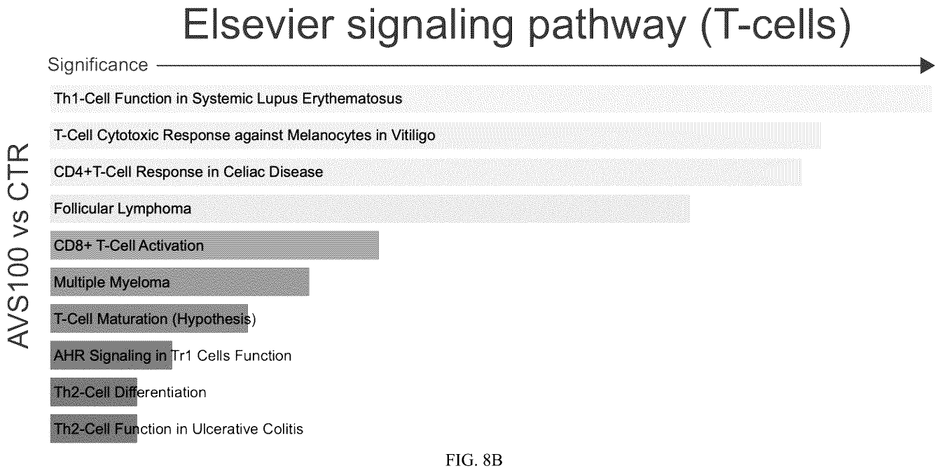

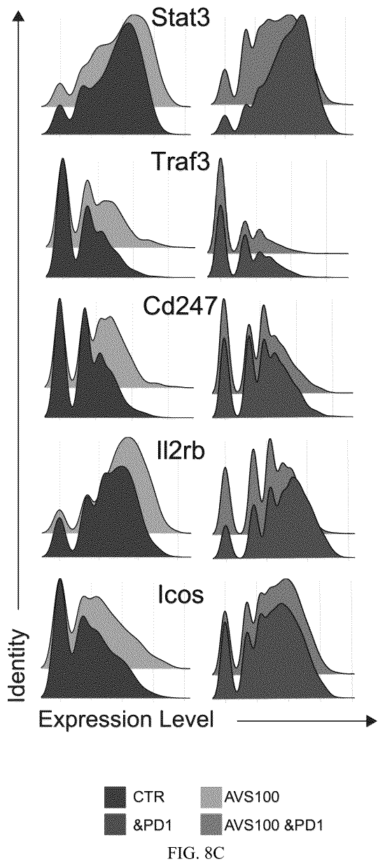

Single-cell transcriptomics analysis shows that AVS100 treatment increases the TAM inflammatory regulatory ratio and T-cell effector functions: To better understand AVS100-induced changes in the tumor microenvironment, the present inventors performed scRNAseq of CD45⁺ sorted immune cells from tumor single-cell suspensions after treatment. Cluster analysis identified a diverse immune cell compartment in which lymphoid cells, including B-cells, T-cells, NK, and NKT cells, grouped together, while myeloid cells, including monocytes, macrophages, and dendritic cells, grouped together (FIG. 7A). Changes in the proportions of different immune cell types after treatment was then analyzed. Macrophages and monocytes were the most abundant cell types, followed by NKT and T-cells, and different treatments did not alter this hierarchy (FIG. 8A).

Seven TAM sub-clusters were determined to identify further how AVS100 treatment affected TAM sub-populations (FIG. 7B) and analyzed the expression of genes that have been associated with the M1 and M2 phenotype in mice. This analysis identified that most TAM subclusters had a mixed M1/M2 signature biased towards proinflammatory (M1-like) or anti-inflammatory (M2-like) phenotypes. Clusters 0, 6, and 7 showed biased expression of M1 genes such as III, Stat1, and Irf1. Clusters 1 and 2 had an M2 bias based on the preferential expression of M2 genes such as Ccl6, Ccl9, Ccl24, Mrc1, Arg1, and Retnla, while cluster 3 had a mixed phenotype characterized by expression of Arg1 and Nos2 prototype M2 and M1 markers respectively (FIG. 7C). The high functional complexity of TAMs has been described by scRNAseq of patient tumors, identifying six major TAM subsets in diverse tumor types. These include Inflammatory, IFN, regulatory, lipid-associated, proliferating, and angiogenic TAMs. Classification of TAM sub-clusters based on this functional phenotype identified cluster 0 as IFN, cluster 1 as lipid-associated, cluster 2 as regulatory, cluster 3 as angiogenic, cluster 4 as proliferative, and cluster 7 as inflammatory TAMs (FIG. 7D).

How the proportion of different TAM sub-clusters changed by treatment was also analyzed. It was observed that Cluster 0, IFN-TAMs were increased by anti-PD1 and AVS100 treatment. This effect was additive as AVS100 plus anti-PD1 combination treatment led to a higher increase. Both AVS100 and anti-PD1 treatment led to a decrease of anti-inflammatory TAM subclusters 2 and 3, and this effect was also additive as a major decrease was observed on AVS100 plus anti-PD1 combination treatment. Cluster 1 showed a reduction by anti-PD1 treatment (FIGS. 7E and 7F).

Also evaluated was how AVS100 treatment regulated gene expression in tumor infiltrating T-cells. Elsevier pathway analysis of genes that showed upregulation in AVS100 vs Control and AVS100 plus anti-PD1 vs anti-PD1 treatment identified upregulation of genes involved in T-cell activation, T-cell effector function, and NFKB signaling by AVS100 treatment (FIG. 8B). Genes upregulated by AVS100 treatment involved in T-cell function are shown in (FIG. 7G), and those belonging to NFKB signaling pathway are shown in (FIG. 7H). Interestingly, Stat4, Nfkb1, Rel, and Il12rb2, which control IFN and NFKB signals, Hcst, Itpr1, and Itgal1, which controls the inositol 3-phosphate/Akt pathway and immune infiltration, were upregulated by AVS100 treatment independently of anti-PD1 therapy (FIG. 7I). Other T-cell related genes showing upregulation by AVS100 only in the absence of anti-PD1 therapy are Stat3, Traf3, Cd247, Il2rb, and Icos (FIG. 8C).

Altogether, scRNAseq analysis confirmed an altered balance towards a proinflammatory tumor microenvironment after AVS100 treatment, mediated by an increase of IFN-TAMs, a decrease of regulatory and angiogenic TAMs, and an increased T-cell effector signature.

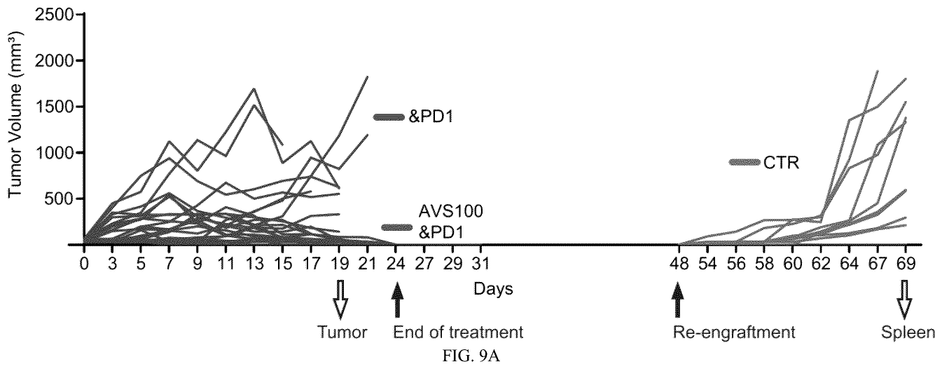

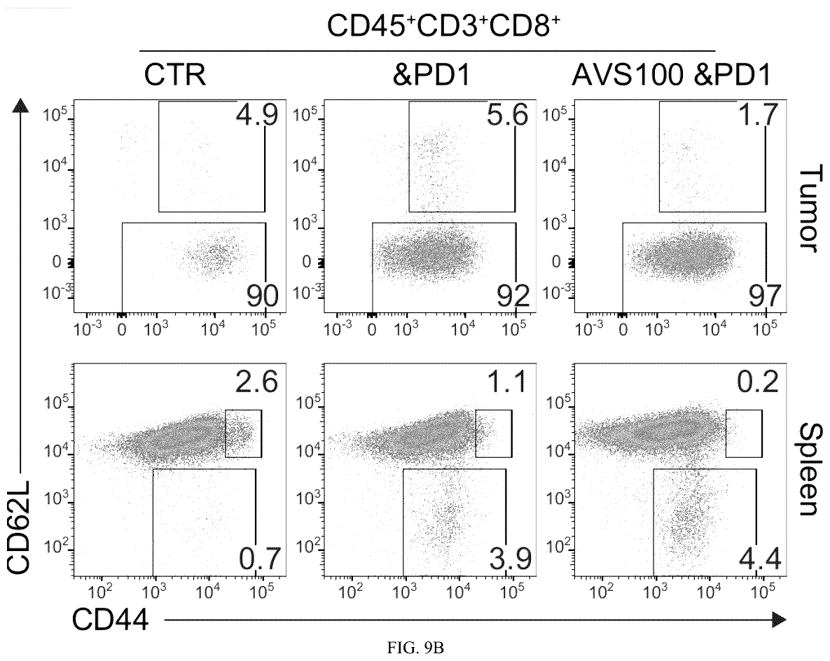

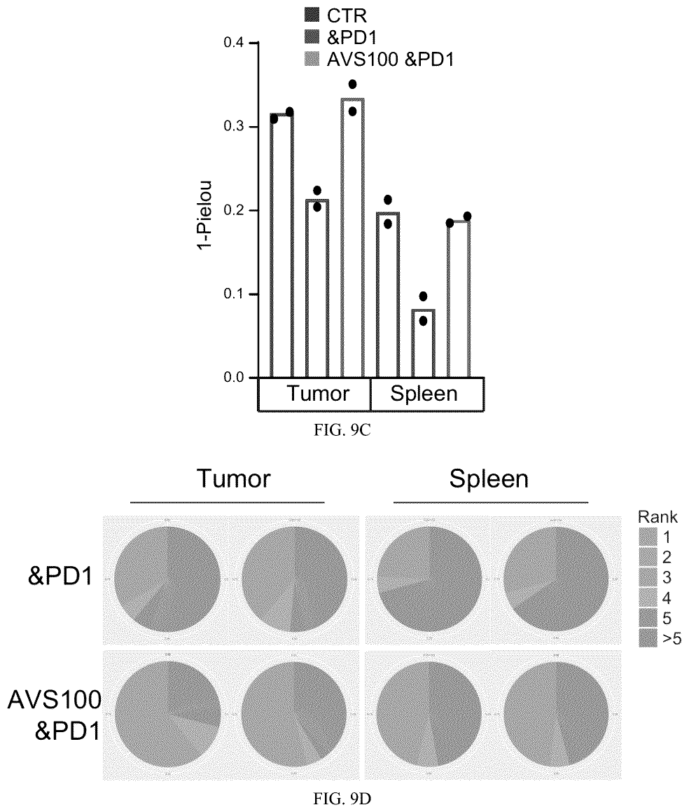

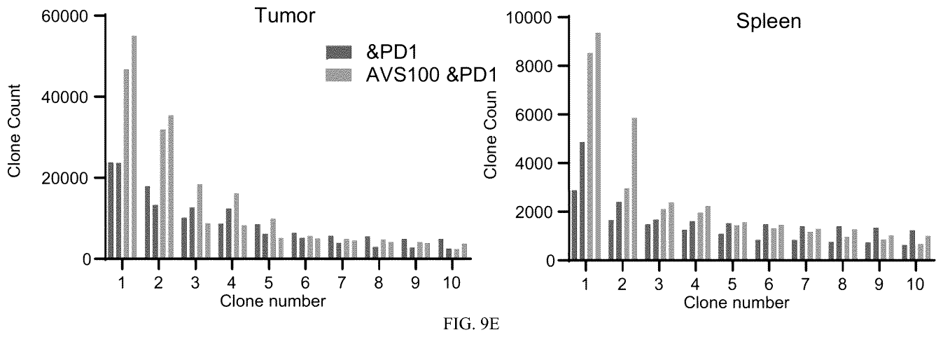

AVS100 treatment amplifies intra-tumoral immunodominant T-cell clones and generates long-term immunity: In vivo treatment of SM1 melanoma tumors with anti-PD1 led to remission of tumors in 61% of mice and treatment with AVS100 plus anti-PD1 combination therapy to responses in 93% of mice (FIG. 5D). After treatment was stopped, tumor growth was monitored for approximately one month in those mice that showed complete elimination of tumors. No tumor regrowth was observed in anti-PD1 or AVS100 plus anti-PD1 treatment groups (FIG. 9A). Then, SM1 tumors were re-implanted in these mice using naïve C57BL/6 mice as controls. It was observed that tumors did not engraft in anti-PD1 or AVS100 plus anti-PD1 treatment groups, while tumor growth was observed in control mice. This indicated that both anti-PD1 and AVS100 plus anti-PD1 therapy elicited long-term antitumoral protection after termination of the treatment.

To evaluate how AVS100 treatment affected the T-cell response elicited by anti-PD1 treatment, we performed TCRseq analysis. As represented in FIG. 9A, CD45⁺ immune cells were isolated from tumors of mice undergoing treatment and CD8 T-cells from spleens of mice that were cured after treatment and had become resistant to a tumor re-challenge. To focus the experiment on the identification of antitumoral T-cells that have migrated from tumors to the periphery, analysis was performed on sorted and pooled central memory (CD62L⁺CD44⁺) and effector memory (CD62L⁻CD44⁺) CD8 T-cells from spleens. Analysis of the phenotype of intratumoral CD8 T-cells revealed that 90-97% of cells had an effector memory phenotype, and only approximately 5% of cells had a central memory phenotype (FIG. 9B). CD8 T-cells from the spleen of cured mice showed an increased proportion of effector memory cells in both anti-PD1 and AVS100 plus anti-PD1 groups compared to control mice, suggesting that amplified T-cells after anti-PD1 therapy could be detected in the spleen of cured mice.

TCRseq was used to identify the abundance of specific T-cell clones and interrogate how treatment affected the diversity of the effector T-cell population. The Pielou index represents the population distribution in which “1” would represent a perfectly even group. Inversely, 1-Pielou would represent the clonality within a population where a higher number represents lower diversity and higher clonality. Anti-PD1 treatment led to reduced clonality in tumor and spleen samples (FIG. 9C), corresponding to the amplification of diverse antitumoral T-cell clones within the effector memory population (35). Interestingly, AVS100 plus anti-PD1 treatment led to higher clonality compared to anti-PD1 treatment alone, which was represented both in the tumor samples and spleens of cured mice. Analysis of the relative proportion of the most abundant clones showed that anti-PD1 treatment had an even distribution of the five most immunodominant clones (FIG. 9D). Interestingly, AVS100 plus anti-PD1 treatment led to a proportional increase of the two to three most immunodominant clones compared to anti-PD1 treatment alone in tumors and spleens. This amplification of immunodominant clones was also observed when measuring absolute T-cell clone counts (FIG. 9E).

These results show that both anti-PD1 treatment alone and AVS100 plus anti-PD1 combination treatment led to the establishment of antitumoral T-cell immunity, conferring long-term protection to a tumor re-challenge. AVS100 plus anti-PD1 combination treatment led to increased amplification of immunodominant T-cell clones underlying its increased antitumoral efficacy.

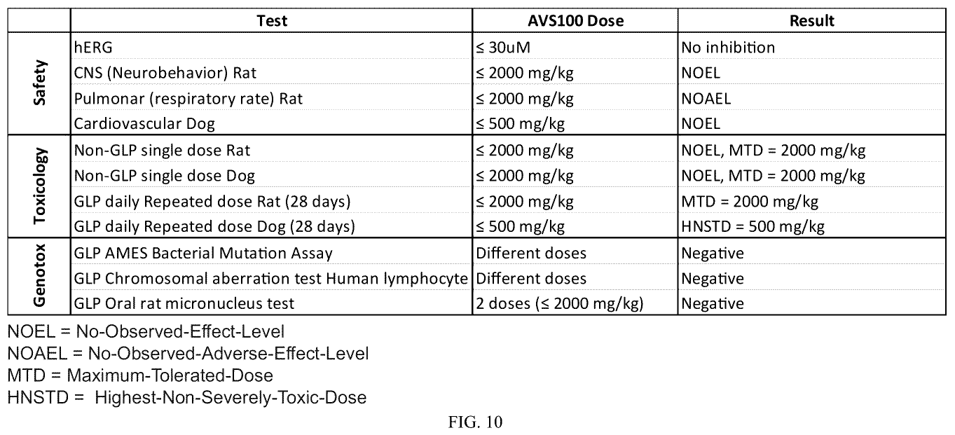

AVS100 safety profile: All the non-GLP and GLP required tests were performed to evaluate AVS100 safety. AVS100 presented a very safe profile, with no alterations of the human cardiac potassium channel function (hERG), CNS behavior, pulmonary or cardiovascular function. In GLP studies, AVS100 showed no mutagenicity or toxicity in rats and dogs (FIG. 10).

AVS100 has an antitumoral effect in mouse models of melanoma and colon cancer and potentiates the effect of immunotherapy. AVS100 inhibited the anti-inflammatory phenotype in macrophages, leading to a proinflammatory tumor microenvironment and facilitating an antitumoral T-cell response.

### Discussion

Checkpoint inhibition with anti-PD1, anti-PDL1, and anti-CTLA4 antibodies reverses the tolerant state of T-cells, triggering an effective antitumoral T-cell response. Several checkpoint inhibitors have been approved for a variety of cancers, including lymphoma, melanoma, non-small cell lung cancer, bladder and kidney cancer, and some head and neck cancers. However, up to 60% of cancer patients remain resistant to immunotherapy in some indications, highlighting the need for combination therapies to disrupt other immunoregulatory pathways simultaneously. In particular, the immunoregulatory microenvironment of tumors is highly determined by myeloid cells, including macrophages and myeloid-derived suppressor cells.

HDAC6 inhibitors are suitable therapy for solid tumors by blocking immunoregulatory functions in TAMs. Data shows that HDAC6 selective inhibitors have a standalone antitumoral effect in melanoma and colon cancer models and also potentiates the antitumoral response of anti-PD1 therapy, leading to the eradication of melanoma and increased anti-PD1 sensitivity in colon cancer. Cured mice did not relapse after termination of the treatment and became resistant to a subsequent tumor challenge. The increased antitumoral effect of combined HDAC6 selective inhibitor plus anti-PD1 therapy was associated with increased amplification of immunodominant T-cell clones within the tumor, supporting a model in which HDAC6 selective inhibitor changes the tumor microenvironment to make it more permissible for antitumoral T-cell expansion and effector functions.

The increased proinflammatory tumor microenvironment was evidenced by increased M1/M2 TAM balance. Previous scRNAseq analyses of TAMs from diverse cancer patients and mice identified seven dominant TAMs with unique phenotypes. Based on this characterization, HDAC6 selective inhibitor led to an increase in proinflammatory IFN-TAMs and a decrease in lipid-associated and regulatory TAMs. Anti-PD1 treatment led to a similar phenotype, and the combination of HDAC6 selective inhibitor plus anti-PD1 treatment had a synergistic effect, highlighting the simultaneous disruption of immunoregulatory signals in T-cells and macrophages. The increase of IFN-TAM by anti-PD1 treatment was expected as this phenotype is generated by T-cell derived IFN-γ signals, which are increased during an antitumoral T-cell response.

Gene expression analysis in tumor infiltrating T-cells revealed an increase of proinflammatory and NFKB signals after HDAC6 selective inhibitor treatment independently of anti-PD1 treatment, including Stat4, Il12r, Nfkb1, and Rel. Some T-cell effector genes were induced by HDAC6 selective inhibitor treatment and could not be further enhanced in combination with anti-PD1. Contrary to the increase in the proinflammatory phenotype in tumor models, analysis of the direct effects of HDAC6 selective inhibitor on macrophages in vitro showed that HDAC6 selective inhibitor exerts a direct role in macrophages by blocking the acquisition of an anti-inflammatory phenotype under M2 polarizing conditions while mildly affecting the acquisition of a M1 phenotype. This suggests that HDAC6 selective inhibitor-mediated inhibition of M2 polarization in macrophages favors a proinflammatory tumor microenvironment in which competing proinflammatory and anti-inflammatory signals coexist in vivo, while these competing signals are not present during in vitro models of macrophage polarization.

STAT6 activation in response to IL-4 signals triggers the alternative activation of macrophages and M2 polarization, and c-Myc is essential for M2 polarization in macrophages. HDAC6 selective inhibitor did not inhibit STAT6 phosphorylation in response to IL-4 stimulation. It has previously been reported that HDAC6 inhibition leads to acetylation of α-tubulin, inefficient nuclear translocation of c-Myc, and its degradation in B-cell lymphoma. c-Myc acetylation also occurs in response to pan-HDAC inhibition, leading to Trail activation and apoptosis in leukemia. The present inventors have observed that HDAC6 selective inhibitor treatment of macrophages increased acetyl-c-Myc levels and reduced total c-Myc levels, as a contributing mechanism for the impaired M2 polarization.

Contrary to non-specific pan-HDAC inhibitors that have received FDA approval and exert direct toxicity to tumor cells, HDAC6 selective inhibitors of the disclosure have minimal toxicity or mutagenic potential. ACY-1215 is an oral bioavailable class I and HDAC6 inhibitor with a ten-fold specificity over HDAC6 than other HDAC isoforms. ACY-1215 has been tested in clinical trials for various indications such as multiple myeloma and lymphoma, and its anti-tumoral actions have been attributed to direct effects in cancer cells. ACY-1215 increased apoptosis in multiple myeloma by inhibiting aggresomal formation and autophagy; it inhibited cell proliferation in cancer cells by disrupting AKT and ERK signaling pathways and activated p53-mediated apoptosis, among other mechanisms. Contrary to HDAC6 selective inhibitors of the disclosure, ACY-1215 reduces proinflammatory functions, most likely due to its direct toxicity on normal lymphoid and myeloid cells and may not share the immune regulatory functions of HDAC6 selective inhibitors of the disclosure.

One particular HDAC6 selective inhibitor of the disclosure, AVS100, has specificity or selectivity for HDAC6 that is at least ten fold more specific than ACY-1215, and that its antitumoral effect is mediated by regulating macrophage function and the intratumoral microenvironment. A similar immune-regulatory role has been described for Nexturastat-A.

HDAC6 selective inhibitors of the disclosure have demonstrated good bioavailability, no mutagenicity and a strong safety profile in rats and dogs making them suitable inter alia for targeting locally advanced or metastatic solid tumors.

As can be seen, HDAC inhibitors of the disclosure have an antitumoral effect as a single agent (i.e., used alone) and improved the efficacy of immune checkpoint inhibition by blocking the immunoregulatory tumor microenvironment and increasing T-cell immunity.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A composition comprising a histone deacetylase (HDAC) inhibitor and a checkpoint inhibitor.

2. The composition according to claim 1, wherein said HDAC inhibitor is an HDAC-6 inhibitor.

3. The composition according to claim 1, wherein said HDAC inhibitor is AMES-negative.

4. The composition according to claim 2, wherein said HDAC inhibitor comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof.

5. The composition according to claim 1, wherein said checkpoint inhibitor comprises a programmed death-1 (PD1) inhibitor, a programmed death ligand-1 (PDL1) inhibitor, a cytotoxic T-lymphocyte-associated protein 4 (CTLA4) inhibitor, a lymphocyte activation gene-3 (LAG-3, or CD223) inhibitor, a T cell immunoreceptor with Ig and ITIM domains (TIGIT) inhibitor, a CD40 inhibitor, an indoleamine 2,3-dioxygenase (IDO) inhibitor, a CCR4 inhibitor, a stimulator of interferon genes (STING) inhibitor, a CD137 inhibitor, a B7-1 inhibitor, a B7-2 inhibitor, or a combination thereof.

6. The composition according to claim 6, wherein said checkpoint inhibitor comprises ipilimumab, nivolumab, pembrolizumab, atezolizumab, avelumab, durvalumab, cemiplimab, or a combination thereof.

7. A method for treating a subject suffering from cancer, said method comprising administering a therapeutically effective amount of a histone deacetylase (HDAC) inhibitor and a checkpoint inhibitor.

8. The method according to claim 7, wherein said histone deacetylase (HDAC) inhibitor and said checkpoint inhibitor are administered as a single composition.

9. The method according to claim 7, wherein said histone deacetylase (HDAC) inhibitor and said checkpoint inhibitor are administered separately.

10. The method according to claim 7, wherein said HDAC inhibitor is an HDAC-6 inhibitor.

11. The method according to claim 10, wherein said HDAC inhibitor is AMES-negative.

12. The method according to claim 7, wherein said HDAC inhibitor comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof.

13. The method according to claim 7, wherein said checkpoint inhibitor comprises a programmed death-1 (PD1) inhibitor, a programmed death ligand-1 (PDL1) inhibitor, a cytotoxic T-lymphocyte-associated protein 4 (CTLA4) inhibitor, or a combination thereof.

14. The method according to claim 13, wherein said checkpoint inhibitor comprises ipilimumab, nivolumab, pembrolizumab, atezolizumab, avelumab, durvalumab, cemiplimab, or a combination thereof.

15. The method according to claim 7, wherein said cancer is a solid tumor cancer.

16. The method according to claim 7, wherein said cancer comprises melanoma, non-Hodgkin's lymphoma, Hodgkin's disease, Ewing's sarcoma, testicular cancer, prostate cancer, ovarian cancer, bladder cancer, larynx cancer, cervical cancer, nasopharynx cancer, breast cancer, colon cancer, pancreatic cancer, head and neck cancer, esophageal cancer, rectal cancer, small-cell lung cancer, non-small cell lung cancer, brain tumors, other CNS neoplasms.

17. A composition comprising a histone deacetylase-6 (HDAC6) inhibitor and a programmed death-1 (PD1) inhibitor.

18. The composition of claim 17, wherein said HDAC6 inhibitor comprises 4-((1-butyl-3-phenylureido)methyl)-N-hydroxybenzamide; 4-((3-(4-(aminomethyl)phenyl)-1-(4-hydroxybutyl)ureido)methyl)-N-hydroxybenzamide; 5-(2-(3,4-dichlorobenzamido)ethyl)-N-hydroxyisoxazole-3-carboxamide; or a combination thereof.

19. The composition of claim 18, wherein said HDAC6 inhibitor comprises 5-(2-(3,4-dichlorobenzamido)ethyl)-N-hydroxyisoxazole-3-carboxamide.

20. The composition of claim 17, wherein said PD1 inhibitor comprises ipilimumab, nivolumab, pembrolizumab, atezolizumab, avelumab, durvalumab, cemiplimab, or a combination thereof.


SEQUENCE LISTING (28 sequences)

SEQ ID NO: 1 - DNA, 24 bp, synthetic construct
  source 1..24: mol_type = other DNA
tccagctaactatccctccactgt

SEQ ID NO: 2 - DNA, 23 bp, synthetic construct
  source 1..23: mol_type = other DNA
ggcccatctgttcatagtcttga

SEQ ID NO: 3 - DNA, 29 bp, synthetic construct
  source 1..29: mol_type = other DNA
accaactattgcttcagcttcagctccac

SEQ ID NO: 4 - DNA, 21 bp, synthetic construct
  source 1..21: mol_type = other DNA
gatccacttccaacccaggtc

SEQ ID NO: 5 - DNA, 22 bp, synthetic construct
  source 1..22: mol_type = other DNA
gagacagggaagtctgaagcac

SEQ ID NO: 6 - DNA, 21 bp, synthetic construct
  source 1..21: mol_type = other DNA
cagcagtagttgctcctcttc

SEQ ID NO: 7 - DNA, 20 bp, synthetic construct
  source 1..20: mol_type = other DNA
atggccttccgtgttcctac

SEQ ID NO: 8 - DNA, 19 bp, synthetic construct
  source 1..19: mol_type = other DNA
cagatgcctgcttcaccac

SEQ ID NO: 9 - DNA, 22 bp, synthetic construct
  source 1..22: mol_type = other DNA
atacaggtgccagagaggtctc

SEQ ID NO: 10 - DNA, 21 bp, synthetic construct
  source 1..21: mol_type = other DNA
cagcttatccttccacgcatg

SEQ ID NO: 11 - DNA, 22 bp, synthetic construct
  source 1..22: mol_type = other DNA
cggtgaaactctggctagacag

SEQ ID NO: 12 - DNA, 22 bp, synthetic construct
  source 1..22: mol_type = other DNA
gcaaaccgtagatgctcaggga

SEQ ID NO: 13 - DNA, 22 bp, synthetic construct
  source 1..22: mol_type = other DNA
cctgctgctttgcctacattgc

SEQ ID NO: 14 - DNA, 22 bp, synthetic construct
  source 1..22: mol_type = other DNA
acacacttggcggttctttcgg

SEQ ID NO: 15 - DNA, 22 bp, synthetic construct
  source 1..22: mol_type = other DNA
ctcttctgcctgctgcactttg

SEQ ID NO: 16 - DNA, 22 bp, synthetic construct
  source 1..22: mol_type = other DNA
atgggctacaggcttgtcactc

SEQ ID NO: 17 - DNA, 22 bp, synthetic construct
  source 1..22: mol_type = other DNA
ttcgcccttagcgtgaagatgg

SEQ ID NO: 18 - DNA, 22 bp, synthetic construct
  source 1..22: mol_type = other DNA
tagtgctccagcagctcgaaga

SEQ ID NO: 19 - DNA, 22 bp, synthetic construct
  source 1..22: mol_type = other DNA
gcctgatctcatagagtctggc

SEQ ID NO: 20 - DNA, 22 bp, synthetic construct
  source 1..22: mol_type = other DNA
tgcatcccagaactagacgtgc

SEQ ID NO: 21 - DNA, 22 bp, synthetic construct
  source 1..22: mol_type = other DNA
cgtctcccatttgactggcaac

SEQ ID NO: 22 - DNA, 23 bp, synthetic construct
  source 1..23: mol_type = other DNA
ccaaatgaatcccacgatggtca

SEQ ID NO: 23 - DNA, 22 bp, synthetic construct
  source 1..22: mol_type = other DNA
gggttgctatcactctctatgc

SEQ ID NO: 24 - DNA, 22 bp, synthetic construct
  source 1..22: mol_type = other DNA
tttcttgtctgttgccgtagtt

SEQ ID NO: 25 - DNA, 22 bp, synthetic construct
  source 1..22: mol_type = other DNA
tgtggcaccaagtacctgctca

SEQ ID NO: 26 - DNA, 22 bp, synthetic construct
  source 1..22: mol_type = other DNA
gcaccttgatgaggttggactc

SEQ ID NO: 27 - DNA, 23 bp, synthetic construct
  source 1..23: mol_type = other DNA
gactttaagggttacctgggttg

SEQ ID NO: 28 - DNA, 21 bp, synthetic construct
  source 1..21: mol_type = other DNA
tcacatgcgccttgatgtctg